SEQUENCE LISTING (1 sequence)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

(19) United States
(12) Patent Application Publication
(10) Pub. No.: US 2017/0360805 A1
(43) Pub. Date: Dec. 21, 2017

(54) MOTOR-ASSOCIATED NEURODEGENERATIVE DISEASE AND METHODS OF TREATMENT

(71) Applicant: Verge Analytics, Inc., San Francisco, CA (US)

(72) Inventors: Jason Chen, San Francisco, CA (US); Alice Zhang, San Francisco, CA (US)

(21) Appl. No.: 15/184,754

(22) Filed: Jun. 16, 2016

(51) Int. Cl. A61K 31/573; A61K 9/00

(52) U.S. Cl. CPC A61K 31/573; A61K 9/0019

(57) ABSTRACT

Disclosed herein are methods and compounds for treating a motor-associated neurodegenerative disease or condition. In some instances, also included herein are methods and compounds for treating a TDP-43 associated neurodegenerative disease or condition (e.g., ALS and frontotemporal dementia) and/or a C9orf72 associated neurodegenerative disease or condition (e.g., ALS and frontotemporal dementia).

### SEQUENCE LISTING

The instant application contains a Sequence Listing which has been submitted in ASCII format via EFS-Web and is hereby incorporated by reference in its entirety. Said ASCII copy, created on Jun. 16, 2016 is named 49393-701.201_SL.txt and is 3.74 kilobytes in size.

### BACKGROUND OF THE DISCLOSURE

The brain is a highly energy-demanding organ that consumes about 20% or more of the total energy produced by an organism. The predominant energy output in the form of ATP comes from oxidative phosphorylation or OXPHOS within the mitochondria. Dysregulation of mitochondrial biogenesis leads to a disruption of neural processing and circuitry, altered neurometabolic coupling, and/or disrupted functional connectivity of neurons, leading to development of neurodegenerative diseases. In addition, protein aggregation and/or genetic mutations further contribute to the etiology of neurodegenerative diseases.

### SUMMARY OF THE DISCLOSURE

Disclosed herein, in certain embodiments, is a method of treating a motor-associated neurodegenerative disease in a subject in need thereof, comprising administering to the subject a therapeutic effective amount of isoflupredone, an isoflupredone derivative, an isoflupredone metabolite, an isoflupredone prodrug, or a pharmaceutically acceptable salt of isoflupredone.

In some embodiments, isoflupredone is administered to the subject in need thereof. In some embodiments, the isoflupredone derivative comprises 21-(1-Ethoxyethoxy)-isoflupredone, isoflupredone acetate or isoflupredone 21-phosphate ester disodium salt. In some embodiments, the isoflupredone derivative comprises isoflupredone acetate.

In some embodiments, the motor-associated neurodegenerative disease is a TDP-43 associated neurodegenerative disease. In some embodiments, the TDP-43 associated neurodegenerative disease comprises amyotrophic lateral sclerosis or frontotemporal dementia.

In some embodiments, the motor-associated neurodegenerative disease is a C9orf72 associated neurodegenerative disease. In some embodiments, the C9orf72 associated neurodegenerative disease comprises amyotrophic lateral sclerosis or frontotemporal dementia.

In some embodiments, the motor-associated neurodegenerative disease comprises a motor neuron disease. In some embodiments, the motor neuron disease comprises amyotrophic lateral sclerosis, primary lateral sclerosis, progressive muscular atrophy, progressive bulbar palsy, or pseudobulbar palsy.

In some embodiments, the motor-associated neurodegenerative disease is amyotrophic lateral sclerosis.

In some embodiments, the motor-associated neurodegenerative disease is frontotemporal dementia.

In some embodiments, isoflupredone, an isoflupredone derivative, an isoflupredone metabolite, an isoflupredone prodrug, or a pharmaceutically acceptable salt of isoflupredone, is formulated for parenteral administration. In some embodiments, isoflupredone, an isoflupredone derivative, an isoflupredone metabolite, an isoflupredone prodrug, or a pharmaceutically acceptable salt of isoflupredone, is formulated for intravenous, intramuscular, or subcutaneous administration.

In some embodiments, the subject is diagnosed with a motor-associated neurodegenerative disease.

In some embodiments, the subject has a mutation in one or more of EIF2AK3, ATF6, C9orf72, SOD1, TARDBP, FUS, COPS4, or TAX1BP1.

In some embodiments, the subject has an elevated level of EIF2AK3 and/or ATF6 relative to a control subject. In some embodiments, the control subject is a subject with a normal level of EIF2AK3 and/or ATF6.

In some embodiments, the treatment with isoflupredone, an isoflupredone derivative, an isoflupredone metabolite, an isoflupredone prodrug, or a pharmaceutically acceptable salt of isoflupredone modulates unfolded protein response pathway or mitochondrial biogenesis pathway.

In some embodiments, the subject is human.

Disclosed herein, in certain embodiments, is a method of treating a TDP-43 associated neurodegenerative disease in a subject in need thereof, comprising administering to the subject a therapeutic effective amount of isoflupredone, an isoflupredone derivative, an isoflupredone metabolite, an isoflupredone prodrug, or a pharmaceutically acceptable salt of isoflupredone.

Disclosed herein, in certain embodiments, is a method of treating a C9orf72 associated neurodegenerative disease in a subject in need thereof, comprising administering to the subject a therapeutic effective amount of isoflupredone, an isoflupredone derivative, an isoflupredone metabolite, an isoflupredone prodrug, or a pharmaceutically acceptable salt of isoflupredone.

Disclosed herein, in certain embodiments, is a method of treating amyotrophic lateral sclerosis in a subject in need thereof, comprising administering to the subject a therapeutic effective amount of isoflupredone, an isoflupredone derivative, an isoflupredone metabolite, an isoflupredone prodrug, or a pharmaceutically acceptable salt of isoflupredone.

Disclosed herein, in certain embodiments, is a method of treating frontotemporal dementia in a subject in need thereof, comprising administering to the subject a therapeutic effective amount of isoflupredone, an isoflupredone derivative, an isoflupredone metabolite, an isoflupredone prodrug, or a pharmaceutically acceptable salt of isoflupredone.

Disclosed herein, in certain embodiments, is a method of treating a motor-associated neurodegenerative disease in a subject in need thereof, comprising administering to the subject a therapeutic effective amount of 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂.

In some embodiments, the method comprises administering to the subject a therapeutic effective amount of 15-deoxy-Δ12,14-prostaglandin J₂.

In some embodiments, the motor-associated neurodegenerative disease is a TDP-43 associated neurodegenerative disease. In some embodiments, the TDP-43 associated neurodegenerative disease comprises amyotrophic lateral sclerosis or frontotemporal dementia.

In some embodiments, the motor-associated neurodegenerative disease is a C9orf72 associated neurodegenerative disease. In some embodiments, the C9orf72 associated neurodegenerative disease comprises amyotrophic lateral sclerosis or frontotemporal dementia.

In some embodiments, the motor-associated neurodegenerative disease comprises a motor neuron disease. In some embodiments, the motor neuron disease comprises amyotrophic lateral sclerosis, primary lateral sclerosis, progressive muscular atrophy, progressive bulbar palsy, or pseudobulbar palsy.

In some embodiments, the motor-associated neurodegenerative disease is amyotrophic lateral sclerosis.

In some embodiments, the motor-associated neurodegenerative disease is frontotemporal dementia.

In some embodiments, 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂ is formulated for parenteral administration. In some embodiments, 15-deoxy-Δ12,14-prostaglandin J2, a 15d-PGJ₂ derivative, a 15d-PGJ₂ metabolite, a 15d-PGJ₂ analog, or a pharmaceutically acceptable salt thereof of 15d-PGJ₂ is formulated for intravenous, intramuscular, or subcutaneous administration.

In some embodiments, the subject is diagnosed with a motor-associated neurodegenerative disease.

In some embodiments, the subject has a mutation in one or more of EIF2AK3, ATF6, C9orf72, SOD1, TARDBP, FUS, COPS4, or TAX1BP1.

In some embodiments, the subject has an elevated level of EIF2AK3 and/or ATF6 relative to a control subject. In some embodiments, the control subject is a subject with a normal level of EIF2AK3 and/or ATF6.

In some embodiments, the treatment with 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂ modulates unfolded protein response pathway or mitochondrial biogenesis pathway.

In some embodiments, the subject is human.

Disclosed herein, in certain embodiments, is a method of treating a TDP-43 associated neurodegenerative disease in a subject in need thereof, comprising administering to the subject a therapeutic effective amount of 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂.

Disclosed herein, in certain embodiments, is a method of treating a C9orf72 associated neurodegenerative disease in a subject in need thereof, comprising administering to the subject a therapeutic effective amount of 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂.

Disclosed herein, in certain embodiments, is a method of treating amyotrophic lateral sclerosis in a subject in need thereof, comprising administering to the subject a therapeutic effective amount of 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂.

Disclosed herein, in certain embodiments, is a method of treating frontotemporal dementia in a subject in need thereof, comprising administering to the subject a therapeutic effective amount of 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a summary of network gene expression changes induced by isoflupredone (left) and medrysone (right) in the Connectivity Map dataset. The intensity of node coloration corresponds to the statistical significance of the differential expression of that gene (green for down-regulated genes, red for up-regulated genes).

FIG. 2A-FIG. 2D show a gene network identified to be associated with ALS pathogenesis. FIG. 2A illustrates a list of genes identified within the ALS-associated gene network (also refers to as M3 network). Genes identified within the gene network (M3 network) are further identified in a study to be downregulated in a SOD1 transgenic mouse model of ALS in the 129 Sv background (FIG. 2B) but is relatively neutral in the C57 background (FIG. 2C) (Nardo, et al., “Transcriptomic indices of fast and slow disease progression in two mouse models of amyotrophic lateral sclerosis,” Brain 136: 3305-3332 (2013)). A separate study shows that genes identified in the gene network are also downregulated in motor neuronal cells extracted from patients with ALS using laser capture microdissection (FIG. 2D) (Highley, et al., “Loss of nuclear TDP-43 in amyotrophic lateral sclerosis (ALS) causes altered expression of splicing machinery and widespread dysregulation of RNA splicing in motor neurons,” Neuropathology and Applied Neurobiology 40: 670-685 (2014)). For FIGS. 2B-2D, the intensity of node coloration corresponds to the statistical significance of the differential expression of the gene (green for down-regulated genes and red for up-regulated genes).

FIG. 3 shows a summary of network gene expression changes induced by 15d-PGJ₂ in the Connectivity Map dataset. The intensity of node coloration corresponds to the statistical significance of the differential expression of that gene (green for down-regulated genes, red for up-regulated genes).

FIG. 4 illustrates the effect of isoflupredone (blue) and other drugs (green) at four concentrations (0.01, 0.1, 1, and 10 μM) on TDP43 stress granule aggregation. Data are shown as mean+SEM (n=6 or 12) as the mean number of fluorescent aggregates per cell. Statistical significance is indicated by *<0.05 and ***<0.0005 as determined by Student's t Test, in comparison to arsenite control (red). Arimoclomol and riluzole were used as positive controls (orange).

FIG. 5 illustrates the activation of M3 network genes in response to isoflupredone (1 μM) in primary rat cortical neurons. The pattern of activation is largely up-regulation (red) with only sparse down-regulation (green), consistent with the predicted effect.

FIG. 6 illustrates the effect of 15d-PGJ2 (blue) and other drugs (green) at four different concentrations (0.01, 0.1, 1, and 10 μM) on TDP43 stress granule aggregation. Data are shown as mean+SEM (n=6 or 12) as the mean number of fluorescent aggregates per cell. Statistical significance is indicated by *<0.05 and ***<0.0005 as determined by Student's t Test, in comparison to arsenite control (red). Arimoclomol and riluzole were used as positive controls (orange).

FIG. 7A-FIG. 7B illustrate the effects of 15d-PGJ2 at four different concentrations (0.01, 0.1, 1, and 10 μM) on glutamate induced neurotoxicity in NSC-34 cells (left-FIG. 7A) and primary rat cortical neurons (right-FIG. 7B). Cell viability was determined by the MTT assay. Data are shown as mean+SEM (n=6 or 12) as percentage of vehicle control (VC). Statistical significance is indicated by *<0.05 and **<0.01 as determined by One-Way ANOVA followed by Dunnett's Multiple Comparison Test, in comparison to glutamate lesion (L or Glu).

### DETAILED DESCRIPTION OF THE DISCLOSURE

Neurodegenerative disease is a group of diseases or conditions characterized by progressive dysfunction, degeneration and death of specific populations of neurons which are often synaptically interconnected. In some embodiments, neurodegenerative disease is further classified into motor-associated neurodegenerative disease, or a disease which affects the motor neurons. In some cases, a motor-associated neurodegenerative disease comprises a TDP-43 associated neurodegenerative disease or motor neuron disease.

In some instances, genetic mutations, a build-up of toxic proteins, and/or a loss of mitochondrial function are associated with a motor-associated neurodegenerative disease. For example, mutations in the C9orf72 gene accounts for about 30% to about 40% of familial amyotrophic lateral sclerosis (ALS) while mutations of the SOD1 gene cause about 15% to about 20% of familial ALS. In addition, protein aggregation and/or misfolding, for example, TDP-43 aggregation, is associated with ALS and frontotemporal lobal degeneration (or frontotemporal dementia). Further, mitochondrial dysfunction, in particular, a defect in the electron transport chain, plays a role, e.g., in the pathogenesis of a motor neuron disease.

In some instances, therapies for a motor-associated neurodegenerative disease focus on alleviating symptoms and/or help to improve a patient's quality of life. For example, riluzole is sometimes used to slow the progression of symptoms in patients with ALS while antidepressants and/or antipsychotics are sometimes used to alleviate some of the behavioral symptoms of frontotemporal dementia.

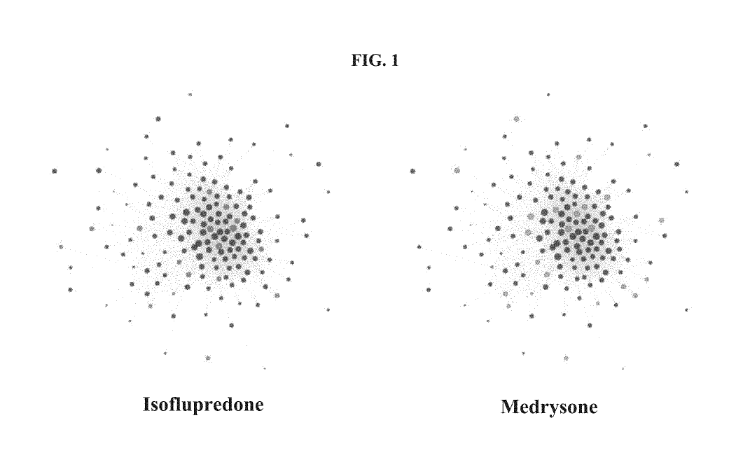

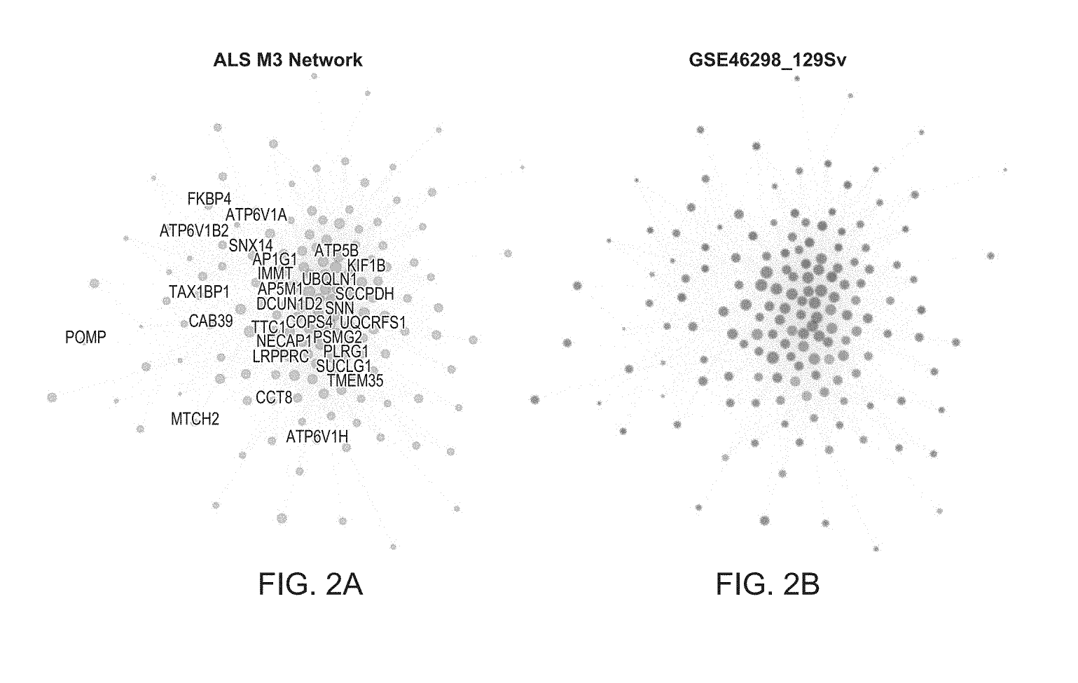

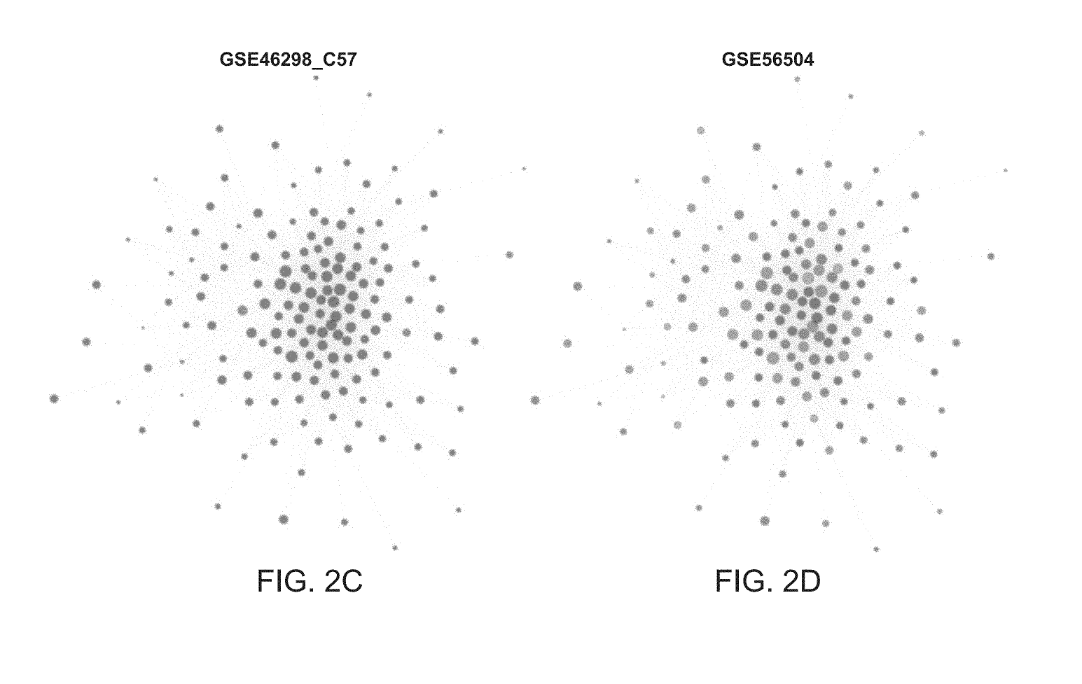

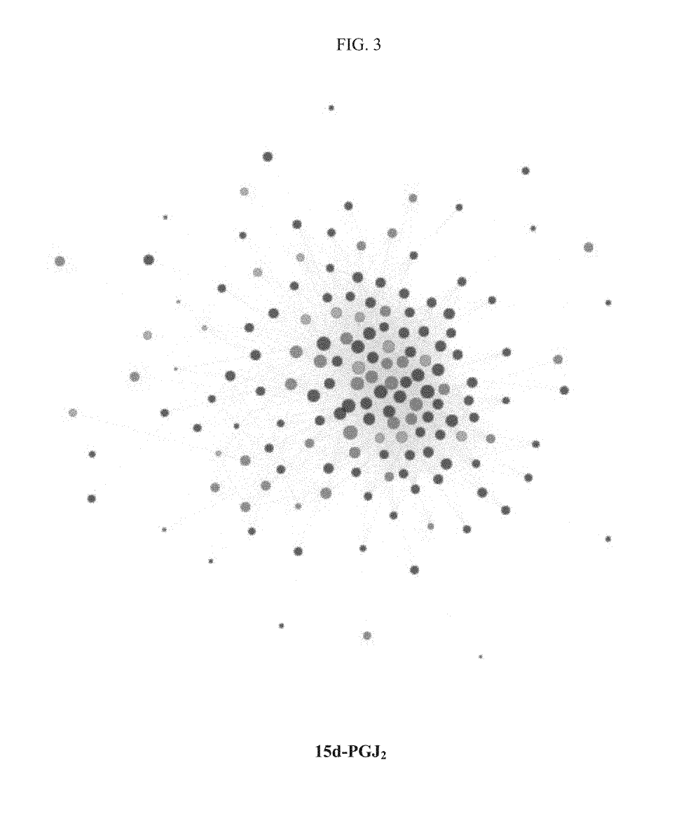

In some instances, isoflupredone and 15-deoxy-Δ12,14-prostaglandin J₂ are shown in a computational analysis to modulate genes associated with one or more of a motor-associated neurodegenerative disease. For example, based on a network pattern matching algorithm and the Connectivity Map database, isoflupredone and 15-deoxy-Δ12,14-prostaglandin J₂ are identified as modulators of one or more genes associated with a motor-associated neurodegenerative disease (e.g., see FIG. 1-FIG. 3). In addition, medrysone is identified to exert an opposite effect in comparison with isoflupredone by the network pattern matching algorithm (FIG. 1).

In some embodiments, disclosed herein is a method of treating a motor-associated neurodegenerative disease in a subject in need thereof, comprising administering to the subject a therapeutic effective amount of isoflupredone, an isoflupredone derivative, an isoflupredone metabolite, an isoflupredone prodrug, or a pharmaceutically acceptable salt thereof of isoflupredone. In some embodiments, a motor-associated neurodegenerative disease comprises a TDP-43 associated neurodegenerative disease, a C9orf72 associated neurodegenerative disease, or motor neuron disease. In some instances, also disclosed herein include a method of treating a TDP-43 associated neurodegenerative disease in a subject in need thereof, comprising administering to the subject a therapeutic effective amount of isoflupredone, an isoflupredone derivative, an isoflupredone metabolite, an isoflupredone prodrug, or a pharmaceutically acceptable salt thereof of isoflupredone. In some instances, additionally disclosed herein include a method of treating a C9orf72 associated neurodegenerative disease in a subject in need thereof, comprising administering to the subject a therapeutic effective amount of isoflupredone, an isoflupredone derivative, an isoflupredone metabolite, an isoflupredone prodrug, or a pharmaceutically acceptable salt thereof of isoflupredone.

In other embodiments, disclosed herein is a method of treating a motor-associated neurodegenerative disease in a subject in need thereof, comprising administering to the subject a therapeutic effective amount of 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂. In some embodiments, a motor-associated neurodegenerative disease comprises a TDP-43 associated neurodegenerative disease, a C9orf72 associated neurodegenerative disease, or motor neuron disease. In some instances, also disclosed herein include a method of treating a TDP-43 associated neurodegenerative disease in a subject in need thereof, comprising administering to the subject a therapeutic effective amount of 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂. In some instances, additionally disclosed herein include a method of treating a C9orf72 associated neurodegenerative disease in a subject in need thereof, comprising administering to the subject a therapeutic effective amount of 15-deoxy-Δ12,14-prostaglandin J₂, a 15d-PGJ₂ derivative, a 15d-PGJ₂ metabolite, a 15d-PGJ₂ analog, or a pharmaceutically acceptable salt thereof of 15d-PGJ₂.

In additional embodiments, disclosed herein include kits comprising isoflupredone and/or 15-deoxy-Δ12,14-prostaglandin J₂, for the treatment of a motor-associated neurodegenerative disease.

### Isoflupredone

Isoflupredone has the chemical name (11β)-9-Fluoro-11,17,21-trihydroxypregna-1,4-diene-3,20-dione. The CAS Registry Number for isoflupredone is 338-95-4. Synonyms for isoflupredone include (8S,9R,10S,11S,13S,14S,17R)-9-fluoro-11,17-dihydroxy-17-(2-hydroxyacetyl)-10,13-dimethyl-6,7,8,9,10,11,12,13,14,15,16,17-dodecahydro-3H-cyclopenta[a]phenanthren-3-one; 1-dehydro-9α-fluorohydrocortisone; 9-fluoroprednisolone.

The chemical structure of isoflupredone is:

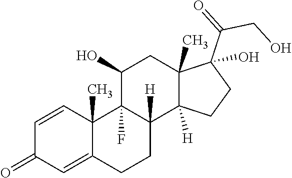

In some embodiments, a derivative of isoflupredone is used in any of the embodiments described herein. Derivatives of isoflupredone include, but are not limited to, metabolites of isoflupredone, prodrugs of isoflupredone and deuterated forms of isoflupredone.

In some embodiments, a derivative of isoflupredone comprises as a prodrug of isoflupredone. A prodrug of isoflupredone is converted into isoflupredone in vivo. Prodrugs are often useful because, in some situations, they are easier to administer than the parent compound. They are, for instances, bioavailable by oral administration whereas the parent is not. In some cases, the prodrug also has improved solubility in pharmaceutical compositions over the parent compound. In some embodiments, the design of a prodrug increases the effective water solubility. In some cases, derivatives of isoflupredone are prepared by forming an ester from any one of the hydroxyl groups of isoflupredone.

An example, without limitation, of a prodrug of isoflupredone is a compound described herein, which is administered as an ester (the “prodrug”) but then is metabolically hydrolyzed to provide isoflupredone. Prodrugs of isoflupredone include, but are not limited to, esters, ethers, carbonates, thiocarbonates, phosphate esters, and sulfonate esters. See for example Design of Prodrugs, Bundgaard, A. Ed., Elseview, 1985 and Method in Enzymology, Rautio et al. “Prodrugs:design and clinical applications” Nature Reviews Drug Discovery, 2008, p. 255-2′70; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group of isoflupredone is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like.

In some embodiments, the hydroxyl at position 21 of isoflupredone is used to form an ester derivative. For example, the hydroxyl at position 21 of isoflupredone is used to form an alkyl ester or a phosphate ester. In some embodiments, the hydroxyl at position 21 of isoflupredone is used to form an acetate derivate (known as isoflupredone acetate). Isoflupredone acetate is also known as 21-acetoxy-9-fluoro-11β,17-dihydroxypregna-1,4-diene-3,20-dione; 9-fluoro-11β,17,21-trihydroxypregna-1,4-diene-3,20-dione 21-acetate; 9α-fluoroprednisolone 21-acetate; 9α-fluoroprednisolone Acetate; NSC 12600; NSC 37977; 9-fluoroprednisolone acetate; Predef; Predef R 2X; U 6013; 9α-Fluoro-11β,17α,21-trihydroxypregna-1,4-diene-3,20-dione 21-Acetate. The CAS Registry Number for isoflupredone acetate is 338-98-7. The chemical structure of isoflupredone acetate is:

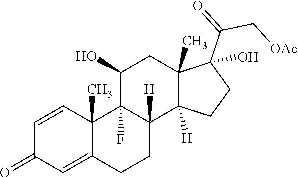

Other ester derivatives of isoflupredone include, but are not limited to:

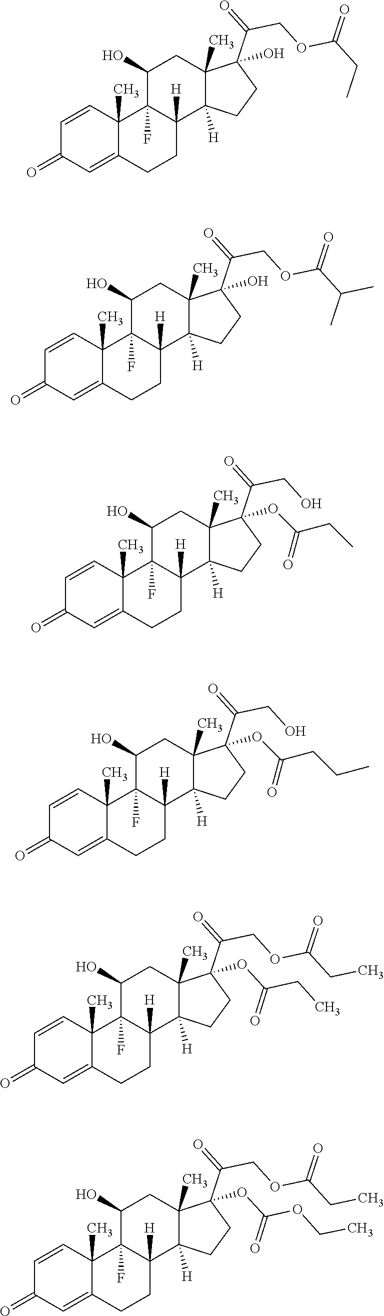

In some embodiments, the hydroxyl at position 21 of isoflupredone is used to form a phosphate derivate. The phosphate ester is known as isoflupredone 21-phosphate ester. In some embodiments, the phosphate ester of isoflupredone has the following structure:

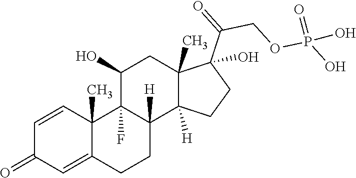

In other embodiments, the phosphate ester of isoflupredone is prepared as a salt form. For example, isoflupredone 21-phosphate ester is prepared as the disodium salt. Isoflupredone 21-phosphate ester disodium salt has the following structure:

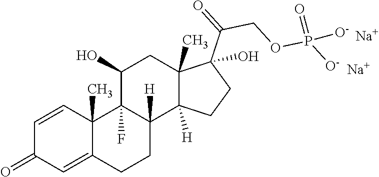

In some embodiments, the phosphate ester of isoflupredone is bioconverted to isoflupredone by alkaline phosphatases.

In some embodiments, an isoflupredone derivative includes, but is not limited to, 21-(1-Ethoxyethoxy)-isoflupredone, which has the following structure:

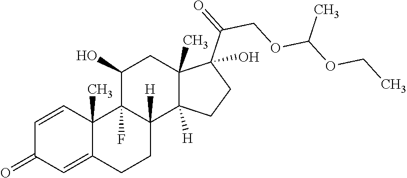

In some embodiments, sites on isoflupredone are susceptible to various metabolic reactions. Incorporation of appropriate substituents on isoflupredone will reduce, minimize or eliminate these metabolic pathways. In specific embodiments, the appropriate substituent(s) to decrease or eliminate the susceptibility of isoflupredone to metabolic reactions is, by way of example only, deuterium atom(s).

In another embodiment, isoflupredone is isotopically labeled (e.g. with deuterium). In some embodiments, isotopically-labeled isoflupredone is identical to isoflupredone but for the fact that one or more atoms are replaced by an atom(s) having an atomic mass or mass number different from the atomic mass or mass number usually found in nature. Examples of isotopes that can be incorporated into isoflupredone include isotopes of hydrogen, carbon, oxygen, and fluorine, such as, for example, ²H, ³H, ¹³C, ¹⁴C, ¹⁸O, ¹⁷O, ¹⁸F. In one aspect, isotopically-labeled isoflupredone, for example those into which radioactive isotopes such as ³H and ¹⁴C are incorporated, is useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, a derivative of isoflupredone is a deuterated version of the compound. In some instances, a deuterated version of the compound comprises at least one, two, three, four, five, six, seven, eight, nine, ten, or more deuterium substitutions. In some cases, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

In some embodiments, deuterated isoflupredone includes isoflupredone-d3 (d2 Major). Isoflupredone-d3 (d2 Major) is also known as (11β)-9-Fluoro-11,17,21-trihydroxypregna-1,4-diene-3,20-dione-d3; 9-Fluoro-11β,17,21-trihydroxy-pregna-1,4-diene-3,20-dione-d3; 9-Fluoroprednisolone-d3; 9α-Fluoro-1-cortisol-d3; Δ-Fluorocortisone-d3; NSC 12174-d3. isoflupredone-d3 has the following structure:

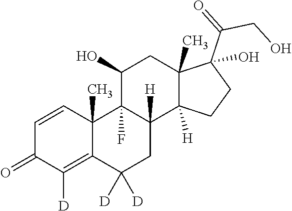

In some embodiments, deuterated isoflupredone includes (8S,9R,10S,11S,13S,14S,17R)-4,6,6-trideuterio-17-(2,2-dideuterio-2-hydroxyacetyl)-9-fluoro-11,17-dihydroxy-10,13-dimethyl-8,11,12,14,15,16-hexahydro-7H-cyclopenta[a]phenanthren-3-one and the following chemical structure:

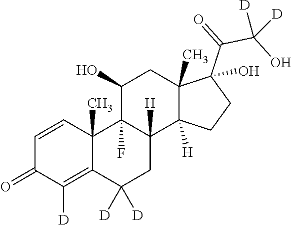

In some cases, a pharmaceutically acceptable salt of isoflupredone, or a derivative of isoflupredone is provided. In some embodiments, a “pharmaceutically acceptable salt” includes a salt with an inorganic base, organic base, inorganic acid, organic acid, or basic or acidic amino acid. Salts of inorganic bases include, for example, alkali metals such as sodium or potassium; alkaline earth metals such as calcium and magnesium or aluminum; and ammonia. Salts of organic bases include, for example, trimethylamine, triethylamine, pyridine, picoline, ethanolamine, diethanolamine, and triethanolamine. Salts of inorganic acids include for example, hydrochloric acid, hydroboric acid, nitric acid, sulfuric acid, and phosphoric acid. Salts of organic acids include for example, formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. Salts of basic amino acids include, for example, arginine, lysine and ornithine. Acidic amino acids include, for example, aspartic acid and glutamic acid.

In some embodiments, exemplary pharmaceutically acceptable salts of isoflupredone or a derivative of isoflupredone include, but are not limited to, isoflupredone 21-phosphate ester disodium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, isoflupredone is used for treating a motor-associated neurodegenerative disease. In some cases, a motor-associated neurodegenerative disease comprises TDP-43 associated neurodegenerative disease, C9orf72 associated neurodegenerative disease, or motor neuron disease. In some cases, isoflupredone is used for treating a TDP-43 associated neurodegenerative disease. In some cases, isoflupredone is used for treating a C9orf72 associated neurodegenerative disease. In some cases, isoflupredone is used for treating a motor neuron disease.

In some embodiments, an isoflupredone derivative is used for treating a motor-associated neurodegenerative disease. In some cases, a derivative of isoflupredone comprises compounds that are derived from or obtained from isoflupredone. In some instances, a derivative improves its solubility, absorption, biological half-life, and the like, or decreases the toxicity of the molecule, eliminate or attenuate any undesirable side effect of isoflupredone, and the like.

In some instances, an isoflupredone derivative used for treating a motor-associated neurodegenerative disease in a subject comprises isoflupredone acetate, isoflupredone 21-phosphate ester disodium salt, or 21-(1-Ethoxyethoxy)-isoflupredone. In some instances, an isoflupredone derivative used for treating a motor-associated neurodegenerative disease in a subject comprises isoflupredone acetate. In some instances, an isoflupredone derivative used for treating a motor-associated neurodegenerative disease in a subject comprises isoflupredone 21-phosphate ester disodium salt. In some instances, an isoflupredone derivative used for treating a motor-associated neurodegenerative disease in a subject comprises 21-(1-Ethoxyethoxy)-isoflupredone.

In some cases, an isoflupredone derivative used for treating a TDP-43 associated neurodegenerative disease comprises isoflupredone acetate, isoflupredone 21-phosphate ester disodium salt, or 21-(1-Ethoxyethoxy)-isoflupredone. In some cases, an isoflupredone derivative used for treating a TDP-43 associated neurodegenerative disease comprises isoflupredone acetate. In additional cases, an isoflupredone derivative used for treating a TDP-43 associated neurodegenerative disease comprises isoflupredone 21-phosphate ester disodium salt. In some cases, an isoflupredone derivative used for treating a TDP-43 associated neurodegenerative disease comprises 21-(1-Ethoxyethoxy)-isoflupredone.

In some embodiments, an isoflupredone derivative used for treating a C9orf72 associated neurodegenerative disease comprises isoflupredone acetate, isoflupredone 21-phosphate ester disodium salt, or 21-(1-Ethoxyethoxy)-isoflupredone. In some cases, an isoflupredone derivative used for treating a C9orf72 associated neurodegenerative disease comprises isoflupredone acetate. In additional cases, an isoflupredone derivative used for treating a C9orf72 associated neurodegenerative disease comprises isoflupredone 21-phosphate ester disodium salt. In some cases, an isoflupredone derivative used for treating a C9orf72 associated neurodegenerative disease comprises 21-(1-Ethoxyethoxy)-isoflupredone.

In some embodiments, isoflupredone comprises an isoflupredone metabolite. In some instances, a metabolite refers to the intermediates and products of isoflupredone that is formed when isoflupredone is metabolized. In additional embodiments, isoflupredone is metabolized upon administration to an organism in need to produce a metabolite that is then used to produce a desired effect, including a desired therapeutic effect. In some instances, a metabolite of isoflupredone is an active metabolite. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, in some instances, enzymes produce specific structural alterations to a compound.

In some embodiments, metabolites of isoflupredone is optionally identified either by administration of compounds to a host and analysis of tissue samples from the host, or by incubation of compounds with hepatic cells in vitro and analysis of the resulting compounds.

In some instances, an isoflupredone used for treating a motor-associated neurodegenerative disease in a subject comprises a metabolite of isoflupredone. In some cases, an isoflupredone for treating a TDP-43 associated neurodegenerative disease comprises a metabolite of isoflupredone. In other cases, an isoflupredone for treating a C9orf72 associated neurodegenerative disease comprises a metabolite of isoflupredone.

In some embodiments, disclosed herein does not include use of an isoflupredone analog for the treatment of a motor-associated neurodegenerative disease. In some instances, an isoflupredone analog refers to compounds that are structurally and functionally similar to, or mimics the effects of, isoflupredone. In some instances, an analog mimics the biological effect of isoflupredone. In other instances, an analog mimics the physical effect of isoflupredone.

In some instances, an isoflupredone used for treating a motor-associated neurodegenerative disease in a subject does not comprise an analog of isoflupredone. In some cases, an isoflupredone for treating a TDP-43 associated neurodegenerative disease does not comprise an analog of isoflupredone. In other cases, an isoflupredone for treating a C9orf72 associated neurodegenerative disease does not comprise an analog of isoflupredone.

In some embodiments, a derivative of isoflupredone does not include a fluorine (F) to hydrogen (H) substitution. For example, isoflupredone comprises a fluorine and the fluorine is not substituted with a hydrogen in a derivative of isoflupredone. In some instances, a derivative of isoflupredone does not comprise prednisolone. In some cases, prednisolone is identified in a computational analysis and the genes modulated by prednisolone contain a different set of characteristics relative to the genes modulated by isoflupredone. In some instances, the set of genes modulated by prednisolone is different relative to the set of genes modulated by isoflupredone. In some instances, the expression levels of the set of genes modulated by prednisolone are different relative to the expression levels of the set of genes modulated by isoflupredone.

In some instances, an isoflupredone used for treating a motor-associated neurodegenerative disease in a subject does not comprise a derivative of isoflupredone including a fluorine to hydrogen substitution. In some cases, an isoflupredone for treating a TDP-43 associated neurodegenerative disease does not comprise a derivative of isoflupredone including a fluorine to hydrogen substitution. In other cases, an isoflupredone for treating a C9orf72 associated neurodegenerative disease does not comprise a derivative of isoflupredone including a fluorine to hydrogen substitution.

### 15-Deoxy-Δ12,14-Prostaglandin J₂

15-Deoxy-Δ12,14-prostaglandin J₂ (15d-PGJ₂) is a product of PGD₂, a prostaglandin that recruits Th2 cells, eosinophils, and basophils. PGD₂ is part of the prostaglandins (PGs) family that also contains the members PGE₂, PGF₂, PGI₂, and thromboxane A₂. In some cases, 15d-PGJ₂ is a metabolite of PGD₂, which involves a sequential conversion of PGD₂ to PGD₂, Δ¹²-PGJ₂, and to 15d-PGJ₂.

In some instances, the cyclopentenone containing 15d-PGJ₂ is proposed to exert anti-inflammatory effects via PPARγ-dependent and PPARγ-independent effects (Scher and Pillinger, “15d-PGD2: The anti-inflammatory prostaglandin?” Clinical Immunology 114: 100-109 (2005); Straus and Glass, “Cyclopentenone prostaglandins: new insights on biological activities and cellular targets,” Medicinal Research Reviews 21: 185-210 (2001); Uchida and Shibata, “15-Deoxy-Δ12,14-prostaglandin J2: an electrophilic trigger of cellular responses,” Chemical Research in Toxicology 21: 138-144 (2008)). In some instances, 15d-PGD2 has been implicated in 1) induction of the antioxidant response by modification of Keap1 in the Keap1-Nrf2 complex; 2) anti-inflammatory activity by covalently binding to IKK, NF-κB, or PPARγ; 3) induces oxidative stress; 4) conjugates to and inhibits the proteasome; and 5) promotes neuronal differentiation and neurite outgrowth; (for a review, see, e.g., Uchida and Shibata, “15-Deoxy-Δ12,14-prostaglandin J2: an electrophilic trigger of cellular responses,” Chemical Research in Toxicology 21: 138-144 (2008)).

In some instances, 15d-PGJ₂ has also been shown to induce a loss of mitochondrial membrane potential and increased mitochondrial ROS production, depletion of GSH, accumulation of ubiquitinated protein, and increased lipid peroxidation (Kondo, et al., “Cyclopentenone prostaglandins as potential inducers of intracellular oxidative stress,” J Biol Chem 276: 12076-12083 (2001). As disclosed above, mitochondrial dysfunction and oxidative stress, in some instances, are associated with many neurodegenerative diseases.

In some cases, 15d-PGJ₂ as well as its precursor PGE2 and PGD₂ have also been shown to be produced in the central nervous system (CNS) and their elevated expressions have been observed in neurodegenerative diseases. Indeed, a study has shown that increased 15d-PGJ₂—like immunoreactivity in spinal cord sections have been observed in ALS patients (Kondo, et al., “15-Deoxy-Delta(12,14)-prostaglandin J(2): the endogenous electrophile that induces neuronal apoptosis,” PNAS 99: 7367-7372 (2002)).

In some embodiments, described here is a method of treating a motor-associated neurodegenerative disease which comprises administering to a subject in need thereof a therapeutic effective amount of 15d-PGJ₂. In some instances, 15d-PGJ₂ has the chemical structure:

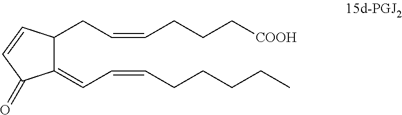 [SMILES: CCCCC/C=C\C=C1\C(=O)C=CC1C/C=C\CCCC(=O)O]

In some instances, also described herein include a 15d-PGJ₂ derivative, a 15d-PGJ₂metabolite, a 15d-PGJ₂ analog, or a pharmaceutically acceptable salt thereof of 15d-PGJ₂. In some cases, described herein includes a pharmaceutically acceptable salt thereof of 15d-PGJ₂. In some cases, a pharmaceutically acceptable salt refers to salts of isoflupredone that have no persistent detrimental effect on the general health of the subject being treated or does not abrogate the biological activity or properties of the compound, and is relatively non-toxic. In some embodiments, a “pharmaceutically acceptable salt” includes a salt with an inorganic base, organic base, inorganic acid, organic acid, or basic or acidic amino acid. Salts of inorganic bases include, for example, alkali metals such as sodium or potassium; alkaline earth metals such as calcium and magnesium or aluminum; and ammonia. Salts of organic bases include, for example, trimethylamine, triethylamine, pyridine, picoline, ethanolamine, diethanolamine, and triethanolamine. Salts of inorganic acids include for example, hydrochloric acid, hydroboric acid, nitric acid, sulfuric acid, and phosphoric acid. Salts of organic acids include for example, formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. Salts of basic amino acids include, for example, arginine, lysine and ornithine. Acidic amino acids include, for example, aspartic acid and glutamic acid.

As discussed above, it should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some instances, a pharmaceutically acceptable salt of 15d-PGJ₂ used for treating a motor-associated neurodegenerative disease in a subject. In some cases, a pharmaceutically acceptable salt of 15d-PGJ₂ used for treating a TDP-43 associated neurodegenerative disease. In some cases, a pharmaceutically acceptable salt of 15d-PGJ₂ used for treating a C9orf72 associated neurodegenerative disease.

In some embodiments, described herein includes a 15d-PGJ₂ derivative. In some cases, a derivative of 15d-PGJ₂ comprises compounds that are derived from or obtained from 15d-PGJ₂. In some instances, a derivative improves its solubility, absorption, biological half-life, and the like, or decreases the toxicity of the molecule, eliminate or attenuate any undesirable side effect of 15d-PGJ₂, and the like.

In some instances, a derivative of 15d-PGJ₂ include an isotopically labeled compound (e.g., with a radioisotope) or by another means, including, but not limited to, the use of chromophores or fluorescent moieties, bioluminescent labels, or chemiluminescent labels. 15d-PGJ₂ derivative include isotopically-labeled compounds, which are identical to the recited structures presented herein, but for the fact that one or more atoms are replaced by an atom having an atomic mass or mass number different from the atomic mass or mass number usually found in nature. Examples of isotopes that are incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In some instances, isotopically-labeled compounds, for example those into which radioactive isotopes such as ³H and ¹⁴C are incorporated, are useful in drug and/or substrate tissue distribution assays.

In some embodiments, a derivative of 15d-PGJ₂ is a deuterated version of the compound. In some instances, a deuterated version of the compound comprises at least one, two, three, four, five, six, seven, eight, nine, ten, or more deuterium substitutions. In some cases, substitution with isotopes such as deuterium affords certain therapeutic advantages resulting from greater metabolic stability, such as, for example, increased in vivo half-life or reduced dosage requirements.

In some instances, a 15d-PGJ₂ derivative is used for treating a motor-associated neurodegenerative disease in a subject. In some cases, a 15d-PGJ₂ derivative is used for treating a TDP-43 associated neurodegenerative disease. In some cases, a 15d-PGJ₂ derivative is used for treating a C9orf72 associated neurodegenerative disease.

In some embodiments, described herein comprises a 15d-PGJ₂ metabolite. In some instances, a metabolite refers to the intermediates and products of 15d-PGJ₂ that is formed when 15d-PGJ₂ is metabolized. In additional embodiments, 15d-PGJ₂ is metabolized upon administration to an organism in need to produce a metabolite that is then used to produce a desired effect, including a desired therapeutic effect. In some instances, a metabolite of 15d-PGJ₂ is an active metabolite. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, in some instances, enzymes produce specific structural alterations to a compound.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In some embodiments, metabolites of 15d-PGJ₂ is optionally identified either by administration of compounds to a host and analysis of tissue samples from the host, or by incubation of compounds with hepatic cells in vitro and analysis of the resulting compounds.

In some instances, a metabolite of 15d-PGJ₂ is used for treating a motor-associated neurodegenerative disease in a subject. In some cases, a metabolite of 15d-PGJ₂ is used for treating a TDP-43 associated neurodegenerative disease. In some cases, a metabolite of 15d-PGJ₂ is used for treating a C9orf72 associated neurodegenerative disease.

In some embodiments, described herein includes a 15d-PGJ₂ analog. In some instances, a 15d-PGJ₂ analog refers to compounds that are structurally and functionally similar to, or mimics the effects of, 15d-PGJ₂. In some instances, an analog mimics the biological effect of 15d-PGJ₂. In other instances, an analog mimics the physical effect of 15d-PGJ₂.

In some instances, an analog of 15d-PGJ₂ is used for treating a motor-associated neurodegenerative disease in a subject. In some cases, an analog of 15d-PGJ₂ is used for treating a TDP-43 associated neurodegenerative disease. In some cases, an analog of 15d-PGJ₂ is used for treating a C9orf72 associated neurodegenerative disease.

### Method of Use

In some embodiments, disclosed herein include methods and kits for treating a motor-associated neurodegenerative disease. In some instances, a method of treating a motor-associated neurodegenerative disease comprises administering to a subject in need thereof a therapeutic effective amount of isoflupredone, an isoflupredone derivative, an isoflupredone metabolite, an isoflupredone prodrug, or a pharmaceutically acceptable salt thereof of isoflupredone. In other instances, a method of treating a motor-associated neurodegenerative disease comprises administering to a subject in need thereof a therapeutic effective amount of 15d-PGJ₂, a 15d-PGJ₂ derivative, a 15d-PGJ₂ metabolite, a 15d-PGJ₂ analog, or a pharmaceutically acceptable salt thereof of 15d-PGJ₂. In some instances, a motor-associated neurodegenerative disease comprises a TDP-43 associated neurodegenerative disease, a C9orf72 associated neurodegenerative disease, or motor neuron disease.

### TDP-43 Associated Neurodegenerative Disease

In some embodiments, the motor-associated neurodegenerative disease is a TDP-43 associated neurodegenerative disease or TDP-43 proteinopathy. Transactive response DNA-binding protein of 43 kDa (TDP-43) is a transcriptional repressor that regulates alternate splicing of the CFTR gene, which encodes an ABC transporter-class ion channel that transports chloride and thiocyanate ions across the cell membrane. In some instances, TDP-43 has also been shown to be a low molecular weight microfilament (hNFL) mRNA-binding protein and as a neuronal activity response factor in the dendrites of hippocampal neurons. In such instances, TDP-43 has been shown to play a role in regulating mRNA stability, transport and local translation in neurons.

In some cases, environmental stress leads to an aggregation of TDP-43. For example, a study has shown that exposure to zinc which promotes oxidative stress leads to an accumulation of insoluble TDP-43 aggregates (Caragounis, et al., “Zinc induces depletion and aggregation of endogenous TDP-43,” Free Radic Biol Med 48: 1152-1161 (2010)). In addition, cultured cells exposed to environmental stress induce TDP-43 to re-locate into stress granules, site of nontranslating mRNAs and factors involved in translation repression and mRNA decay (Cohen, et al., “TDP-43 functions and pathogenic mechanisms implicated in TDP-43 proteinopathies,” Trends Mol Med 17(11):659-667 (2011)).

In some instances, accumulation of TDP-43 aggregates has been linked to neurodegenerative diseases, e.g., motor-associated neurodegenerative diseases. For example, TDP-43 is a component of tau-negative and ubiquitin-positive inclusions that characterize amyotrophic lateral sclerosis (ALS) and frontotemporal lobal degeneration (FTLD). In addition, mutations within the TARDBP gene, which encodes the TDP-43 protein, have also been observed in ALS and FTLD patients, and have been linked to disease pathogenesis (Pesiridis, et al., “Mutations in TDP-43 link glycine-rich domain functions to amyotrophic lateral sclerosis,” Hum Mol Genet. 18(R2):R156-R162 (2009)).

In some embodiments, a TDP-43-associated neurodegenerative disease or TDP-43 proteinopathy described herein refers to a neurodegenerative disease or condition characterized by the misfolding and/or aggregation of TDP-43 protein in the neurons and/or one or more mutations in the TDP-43 protein. In some instances, TDP-43 aggregates is located in the motor cortex, brainstem, spinal cord, prefrontal neocortex, precerebellar nuclei, red nucleus, midbrain structure involving motor control, postcentral neocortex, striatum, temporal lobe, or a combination thereof. In some instances, TDP-43 aggregates are located in the motor cortex and then radiate down toward the spinal cord and radiate forward toward the frontal cortices. In some embodiments, a TDP-43-associated neurodegenerative disease or TDP-43 proteinopathy described herein refers to a neurodegenerative disease or condition characterized by a TDP-43 aggregate at the motor cortex, brainstem, spinal cord, prefrontal neocortex, precerebellar nuclei, red nucleus, midbrain structure involving motor control, postcentral neocortex, striatum, temporal lobe, or a combination thereof.

In some embodiments, one or more mutations are associated with the TDP-43 protein. In some instances, the one or more mutations in the TDP-43 protein include, but are not limited to, amino acid position 12, 27, 29, 66, 90, 104, 137, 169, 214, 263, 267, 287, 290, 294, 295, 298, 315, 321, 331, 332, 335, 337, 343, 345, 348, 352, 361, 363, 366, 374, 379, 382, 383, 390, 393 or a combination thereof. In some instances, the one or more mutations include, but are not limited to, amino acid position N12, L27, S29, A66, A90, S104, K137, D169, Y214, K263, N267, G287, G290, G294, G295, G298, A315, A321, Q331, S332, G335, M337, Q343, N345, G348, N352, R361, P363, A366, Y374, S379, A382, 1383, N390, S393 or a combination thereof. In some instances, the one or more mutations include, but are not limited to, A90V, D169G, K263E, N267S, G287S, G290A, G294A, G294V, G295S, G295R, G298S, A315T, A321G, A321V, Q331K, S332N, G335D, M337V, Q343R, N345K, G348C, G348V, N352S, R361S, P363A, Y374X (in which X in some cases comprise a STOP codon), S379P, S379C, A382T, A382P, I383V, N390D, N390S, S393L or a combination thereof. In some instances, one or more mutations associated with the TDP-43 protein include, but are not limited to, mutations at amino acid residue position M337, N345, 1383 or a combination thereof. In some instances, the amino acid position is according to SEQ ID NO: 1 (TDP-43; Accession number: NP_031401.1).

In some embodiments, disclosed herein is a method of treating a TDP-43-associated neurodegenerative disease or TDP-43 proteinopathy, which comprises administering to a subject in need thereof a therapeutic effective amount of isoflupredone, an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some embodiments, disclosed herein is a method of treating a TDP-43-associated neurodegenerative disease or TDP-43 proteinopathy, which comprises administering to a subject in need thereof a therapeutic effective amount of isoflupredone acetate. In some embodiments, disclosed herein is a method of treating a TDP-43-associated neurodegenerative disease or TDP-43 proteinopathy, which comprises administering to a subject in need thereof a therapeutic effective amount of isoflupredone 21-phosphate ester disodium salt. In some embodiments, disclosed herein is a method of treating a TDP-43-associated neurodegenerative disease or TDP-43 proteinopathy, which comprises administering to a subject in need thereof a therapeutic effective amount of 21-(1-Ethoxyethoxy)-isoflupredone. In some embodiments, a TDP-43-associated neurodegenerative disease or TDP-43 proteinopathy comprises ALS and frontotemporal dementia.

In some embodiments, disclosed herein is a method of treating a TDP-43-associated neurodegenerative disease or TDP-43 proteinopathy, which comprises administering to a subject in need thereof a therapeutic effective amount of 15d-PGJ₂, a 15d-PGJ₂ derivative, a 15d-PGJ₂ metabolite, a 15d-PGJ₂ analog, or a pharmaceutically acceptable salt thereof of 15d-PGJ₂. In some embodiments, a TDP-43-associated neurodegenerative disease or TDP-43 proteinopathy comprises ALS and frontotemporal dementia.

### C9orf72 Associated Neurodegenerative Disease

In some embodiments, the motor-associated neurodegenerative disease is a C9orf72 associated neurodegenerative disease. Chromosome 9 Open Reading Frame 72 (C9orf72) encodes a protein involved in the regulation of endosomal trafficking, RNA production, and/or protein production. In some instances, a mutation within the C9orf72 gene has been linked to a neurodegenerative disease such as frototemporal dementia (FTD) and/or amyotrophic lateral sclerosis (ALS). In some instances, the mutation of the C9orf72 gene comprises a hexanucleotide repeat expansion of about six consecutive nucleotides GGGGCC. In some instances, the hexanucleotide repeat expansion comprises more than about 30, 50, 100, 200, 300, 400, or 500 repeats. In some instances, an individual who does not suffer from a neurodegenerative disease comprises about 30 hexanucleotide repeats.

In some embodiments, a C9orf72-associated neurodegenerative disease described herein refers to a neurodegenerative disease or condition characterized by one or more mutations within the C9orf72 gene or the C9orf72 protein. In some instances, the one or more mutations of the C9orf72 gene comprise a hexaucleotide repeat expansion of the nucleotide GGGGCC. In some embodiments, a C9orf72-associated neurodegenerative disease described herein refers to a neurodegenerative disease or condition characterized by one or more of the hexaucleotide repeat expansion of the nucleotide GGGGCC within the C9orf72 gene.

In some embodiments, disclosed herein is a method of treating a C9orf72-associated neurodegenerative disease, which comprises administering to a subject in need thereof a therapeutic effective amount of isoflupredone, an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some embodiments, disclosed herein is a method of treating a C9orf72-associated neurodegenerative disease, which comprises administering to a subject in need thereof a therapeutic effective amount of isoflupredone acetate. In some embodiments, disclosed herein is a method of treating a C9orf72-associated neurodegenerative disease, which comprises administering to a subject in need thereof a therapeutic effective amount of isoflupredone 21-phosphate ester disodium salt. In some embodiments, disclosed herein is a method of treating a C9orf72-associated neurodegenerative disease, which comprises administering to a subject in need thereof a therapeutic effective amount of 21-(1-Ethoxyethoxy)-isoflupredone. In some embodiments, a C9orf72-associated neurodegenerative disease comprises ALS and frontotemporal dementia.

In some embodiments, disclosed herein is a method of treating a C9orf72-associated neurodegenerative disease, which comprises administering to a subject in need thereof a therapeutic effective amount of 15d-PGJ₂, a 15d-PGJ₂ derivative, a 15d-PGJ₂ metabolite, a 15d-PGJ₂ analog, or a pharmaceutically acceptable salt thereof of 15d-PGJ₂. In some embodiments, a C9orf72-associated neurodegenerative disease comprises ALS and frontotemporal dementia.

### Amyotrophic Lateral Sclerosis

Amyotrophic lateral sclerosis (ALS), also called Lou Gehrig's disease, Charcot's disease or classical motor neuron disease, is a neurological disease that leads to progressive loss of motor function and death. In some instances, ALS is referred to as a TDP-43 associated neurodegenerative disease due to the presence of TDP-43 aggregates. In some instances, ALS is referred to as a motor neuron disease. In some instances, ALS is characterized by stiff muscles, muscle twitching, and gradually worsening weakness due to muscles decreasing in size, leading to dyspnea (or difficulty in breathing) and dysphagia (or difficulty or discomfort in swallowing).

In some cases, ALS is further categorized as sporadic ALS, familial-type ALS (FALS), and ALS-parkinsonism-dementia complex (ALS-PDC). In some instances, sporadic ALS comprises about 90-95% of the ALS patient population and does not comprise a genetically inherited component. In some cases, familial-type ALS, which makeups the remainder 5% of the population, comprises an associated genetic dominant inheritance factor. In additional cases, ALS-PDC is characterized by the signs and symptoms of ALS, the pattern movement abnormalities associated with parkinsonism, and a progressive loss of intellectual function (dementia).

In some instances, mutations in several genes have been correlated to ALS pathogenesis. For example, about 20% of patients with familial ALS and about 5% of patients with sporadic ALS have mutations in the gene encoding the antioxidant enzyme Cu/Zn superoxide dismutase 1 (SOD1). In addition, mutations in TARDBP and FUS genes have also been observed in familial ALS. Further, mutations in chromosome 9 (C9orf72) accounts, for example, about 30% to about 40% of familial ALS. As discussed above, chromosome 9 Open Reading Frame 72 (C9orf72) encodes a protein involved in the regulation of endosomal trafficking. In some cases, expansion of a hexanucleotide repeat in non-coding sequence between alternate 5′ exons in transcripts from this gene is associated with 9p-linked ALS and FTD.

In some instances, disclosed herein is a method of treating ALS which comprises administering to a subject in need thereof a therapeutic effective amount of isoflupredone, an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some embodiments, disclosed herein is a method of treating ALS which comprises administering to a subject in need thereof a therapeutic effective amount of isoflupredone acetate. In some embodiments, disclosed herein is a method of treating ALS which comprises administering to a subject in need thereof a therapeutic effective amount of isoflupredone 21-phosphate ester disodium salt. In some embodiments, disclosed herein is a method of treating ALS which comprises administering to a subject in need thereof a therapeutic effective amount of 21-(1-Ethoxyethoxy)-isoflupredone.

In some cases, disclosed herein is a method of treating ALS which comprises administering to a subject in need thereof a therapeutic effective amount of 15d-PGJ₂, a 15d-PGJ₂ derivative, a 15d-PGJ₂ metabolite, a 15d-PGJ₂ analog, or a pharmaceutically acceptable salt thereof of 15d-PGJ₂. In some cases, disclosed herein is a method of treating ALS which comprises administering to a subject in need thereof a therapeutic effective amount of a 15d-PGJ₂ derivative, a metabolite, an analog, or a pharmaceutically acceptable salt thereof.

In some instances, a FDA approved treatment for ALS is riluzole. In some instances, riluzole is further used in combination with isoflupredone. In some instances, described herein is a combination therapy which comprises administration of isoflupredone with riluzole for the treatment of ALS. In some cases, also disclosed herein includes an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some instances, described herein is a combination therapy which comprises administration of isoflupredone acetate with riluzole for the treatment of ALS. In some instances, described herein is a combination therapy which comprises administration of isoflupredone 21-phosphate ester disodium salt with riluzole for the treatment of ALS. In some instances, described herein is a combination therapy which comprises administration of 21-(1-Ethoxyethoxy)-isoflupredone with riluzole for the treatment of ALS.

In some instances, described herein is a combination therapy which comprises administration of 15d-PGJ₂ with riluzole for the treatment of ALS. In some cases, also disclosed herein includes a 15d-PGJ₂ derivative, a 15d-PGJ₂ metabolite, a 15d-PGJ₂ analog, or a pharmaceutically acceptable salt thereof of 15d-PGJ₂. In some cases, described herein is a combination therapy which comprises administration of riluzole with a 15d-PGJ₂ derivative, a 15d-PGJ₂ metabolite, a 15d-PGJ₂ analog, or a pharmaceutically acceptable salt thereof of 15d-PGJ₂ for the treatment of ALS.

### Frontotemporal Dementia

In some embodiments, a TDP-43-associated neurodegenerative disease or TDP-43 proteinopathy comprises frontotemporal dementia. Frontotemporal dementia (FTD) (or Pick's disease, frontotemporal disorder, frontotemporal degeneration, or frontal lobe disorder) is the clinical presentation of frontotemporal lobar degeneration (FTLD), which is characterized by progressive neuronal loss predominantly involving the frontal and/or temporal lobes, and typical loss of over 70% of spindle neurons, while other neuron types remain intact. In some instances, signs and symptoms of FTD include significant changes in social and personal behavior, apathy, blunting of emotions, and deficits in both expressive and receptive language. In some cases, signs and symptoms are classified based on the functions of the frontal and temporal lobes, such as behavioral variant FTD (bvFTD), semantic dementia (SD), and progressive nonfluent aphasia (PNFA). In some instances, behavioral variant FTD (bvFTD) is characterized by changes in social behavior and conduct, with loss of social awareness and poor impulse control. In some instances, semantic dementia (SD) is characterized by the loss of semantic understanding, resulting in impaired word comprehension, although speech remains fluent and grammatically faultless. In some instances, progressive nonfluent aphasia (PNFA) is characterized by progressive difficulties in speech production. In some instances, a subgroup of patients with ALS further develops frontotemporal dementia (FTD).

In some instances, disclosed herein is a method of treating frontotemporal dementia which comprises administering to a subject in need thereof a therapeutic effective amount of isoflupredone, an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some embodiments, disclosed herein is a method of treating frontotemporal dementia which comprises administering to a subject in need thereof a therapeutic effective amount of isoflupredone acetate. In some embodiments, disclosed herein is a method of treating frontotemporal dementia which comprises administering to a subject in need thereof a therapeutic effective amount of isoflupredone 21-phosphate ester disodium salt. In some embodiments, disclosed herein is a method of treating frontotemporal dementia which comprises administering to a subject in need thereof a therapeutic effective amount of 21-(1-Ethoxyethoxy)-isoflupredone.

In some instances, disclosed herein is a method of treating frontotemporal dementia which comprises administering to a subject in need thereof a therapeutic effective amount of 15d-PGJ₂, a 15d-PGJ₂ derivative, a 15d-PGJ₂ metabolite, a 15d-PGJ₂ analog, or a pharmaceutically acceptable salt thereof of 15d-PGJ₂. In some cases, disclosed herein is a method of treating frontotemporal dementia which comprises administering to a subject in need thereof a therapeutic effective amount of a 15d-PGJ₂ derivative, a 15d-PGJ₂ metabolite, a 15d-PGJ₂ analog, or a pharmaceutically acceptable salt thereof of 15d-PGJ₂.

### Motor Neuron Disease

In some embodiments, a motor-associated neurodegenerative disease is a motor neuron disease. In some embodiments, motor neuron disease (MND) is a group of progressive neurological disorders that destroy motor neurons, the cells that control essential voluntary muscle activity such as speaking, walking, breathing, and swallowing. In some embodiments, MND is classified according to whether they are inherited or sporadic, and to whether degeneration affects upper motor neurons, lower motor neurons, or both. In some embodiments, motor neuron disease comprises amyotrophic lateral sclerosis (ALS), primary lateral sclerosis, progressive muscular atrophy, progressive bulbar palsy, or pseudobulbar palsy.

In some embodiments, disclosed herein, is a method of treating motor neuron disease in a subject in need thereof, comprising administering to the subject a therapeutic effective amount of isoflupredone, an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some embodiments, disclosed herein, is a method of treating motor neuron disease in a subject in need thereof, comprising administering to the subject a therapeutic effective amount of isoflupredone acetate. In some embodiments, disclosed herein, is a method of treating motor neuron disease in a subject in need thereof, comprising administering to the subject a therapeutic effective amount of isoflupredone 21-phosphate ester disodium salt. In some embodiments, disclosed herein, is a method of treating motor neuron disease in a subject in need thereof, comprising administering to the subject a therapeutic effective amount of 21-(1-Ethoxyethoxy)-isoflupredone.

In some embodiments, disclosed herein, is a method of treating motor neuron disease in a subject in need thereof, comprising administering to the subject a therapeutic effective amount of 15d-PGJ₂. In some cases, also disclosed herein includes a 15d-PGJ₂ derivative, a 15d-PGJ₂ metabolite, a 15d-PGJ₂ analog, or a pharmaceutically acceptable salt thereof of 15d-PGJ₂. In some cases, disclosed herein, is a method of treating motor neuron disease in a subject in need thereof, comprising administering to the subject a therapeutic effective amount of a 15d-PGJ₂ derivative, a 15d-PGJ₂ metabolite, a 15d-PGJ₂ analog, or a pharmaceutically acceptable salt thereof of 15d-PGJ₂.

### Primary Lateral Sclerosis

In some embodiments, motor neuron disease is primary lateral sclerosis. Primary lateral sclerosis is a disease of the upper motor neurons. In some instances, primary lateral sclerosis affects the upper motor neurons of the arms, legs, and face. For example, this occurs when specific nerve cells in the motor regions of the cerebral cortex gradually degenerate, causing the movements to be slow and effortful. In some cases, the disease affects the legs first, followed by the body trunk, arms and hands, and, finally, the bulbar muscles. In some cases, affected legs and arms become stiff, clumsy, slow and weak, leading to an inability to walk or carry out tasks requiring fine hand coordination. In some instances, individuals experience a pseudobulbar affect and an overactive startle response.

In some embodiments, disclosed herein, is a method of treating primary lateral sclerosis in a subject in need thereof, comprising administering to the subject a therapeutic effective amount of isoflupredone, an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some embodiments, disclosed herein, is a method of treating primary lateral sclerosis in a subject in need thereof, comprising administering to the subject a therapeutic effective amount of isoflupredone acetate. In some embodiments, disclosed herein, is a method of treating primary lateral sclerosis in a subject in need thereof, comprising administering to the subject a therapeutic effective amount of isoflupredone 21-phosphate ester disodium salt. In some embodiments, disclosed herein, is a method of treating primary lateral sclerosis in a subject in need thereof, comprising administering to the subject a therapeutic effective amount of 21-(1-Ethoxyethoxy)-isoflupredone.

In some embodiments, disclosed herein is a method of treating primary lateral sclerosis in a subject in need thereof, comprising administering to the subject a therapeutic effective amount of 15d-PGJ₂. In some cases, also disclosed herein includes a 15d-PGJ₂ derivative, a 15d-PGJ₂ metabolite, a 15d-PGJ₂ analog, or a pharmaceutically acceptable salt thereof of 15d-PGJ₂. In some cases, disclosed herein, is a method of treating primary lateral sclerosis in a subject in need thereof, comprising administering to the subject a therapeutic effective amount of a 15d-PGJ₂ derivative, a 15d-PGJ₂ metabolite, a 15d-PGJ₂ analog, or a pharmaceutically acceptable salt thereof of 15d-PGJ₂.

### Progressive Muscular Atrophy

In some embodiments, the motor neuron disease is progressive muscular atrophy. In some embodiments, progressive muscular atrophy is a disease that affects only lower motor neurons in the spinal cord. In some instances, symptoms and signs include weakness seen first in the hands and then spreading into the lower body, where it can be severe. In some cases, symptoms include, muscle wasting, clumsy hand movements, fasciculations, and muscle cramps. In some cases, the trunk muscles and respiration becomes affected. In some instances, this disease further progresses into ALS.

In some embodiments, disclosed herein is a method of treating progressive muscular atrophy in a subject in need thereof, comprising administering to the subject a therapeutic effective amount of isoflupredone, an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some embodiments, disclosed herein, is a method of treating progressive muscular atrophy in a subject in need thereof, comprising administering to the subject a therapeutic effective amount of isoflupredone acetate. In some embodiments, disclosed herein, is a method of treating progressive muscular atrophy in a subject in need thereof, comprising administering to the subject a therapeutic effective amount of isoflupredone 21-phosphate ester disodium salt. In some embodiments, disclosed herein, is a method of treating progressive muscular atrophy in a subject in need thereof, comprising administering to the subject a therapeutic effective amount of 21-(1-Ethoxyethoxy)-isoflupredone.

In some embodiments, disclosed herein is a method of treating progressive muscular atrophy in a subject in need thereof, comprising administering to the subject a therapeutic effective amount of 15d-PGJ₂. In some cases, also disclosed herein includes a 15d-PGJ₂ derivative, a 15d-PGJ₂ metabolite, a 15d-PGJ₂ analog, or a pharmaceutically acceptable salt thereof of 15d-PGJ₂. In some cases, disclosed herein is a method of treating progressive muscular atrophy in a subject in need thereof, comprising administering to the subject a therapeutic effective amount of a 15d-PGJ₂ derivative, a 15d-PGJ₂ metabolite, a 15d-PGJ₂ analog, or a pharmaceutically acceptable salt thereof of 15d-PGJ₂.

### Progressive Bulbar Palsy

In some embodiments, the motor neuron disease is progressive bulbar palsy. In some embodiments, progressive bulbar palsy is a disease in which the lowest motor neurons of the brain stem are most affected, causing slurred speech and difficulty chewing and swallowing. In other cases, mildly abnormal signs occur in the arms and legs.

In some embodiments, disclosed herein is a method of treating progressive bulbar palsy in a subject in need thereof, comprising administering to the subject a therapeutic effective amount of isoflupredone, an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some embodiments, disclosed herein, is a method of treating progressive bulbar palsy in a subject in need thereof, comprising administering to the subject a therapeutic effective amount of isoflupredone acetate. In some embodiments, disclosed herein, is a method of treating progressive bulbar palsy in a subject in need thereof, comprising administering to the subject a therapeutic effective amount of isoflupredone 21-phosphate ester disodium salt. In some embodiments, disclosed herein, is a method of treating progressive bulbar palsy in a subject in need thereof, comprising administering to the subject a therapeutic effective amount of 21-(1-Ethoxyethoxy)-isoflupredone.

In some embodiments, disclosed herein is a method of treating progressive bulbar palsy in a subject in need thereof, comprising administering to the subject a therapeutic effective amount of 15d-PGJ₂. In some cases, also disclosed herein includes a 15d-PGJ₂ derivative, a 15d-PGJ₂ metabolite, a 15d-PGJ₂ analog, or a pharmaceutically acceptable salt thereof of 15d-PGJ₂. In some cases, disclosed herein is a method of treating progressive bulbar palsy in a subject in need thereof, comprising administering to the subject a therapeutic effective amount of a 15d-PGJ₂ derivative, a 15d-PGJ₂ metabolite, a 15d-PGJ₂ analog, or a pharmaceutically acceptable salt thereof of 15d-PGJ₂.

### Pseudobulbar Palsy

In some embodiments, the motor neuron disease is pseudobulbar palsy. In some embodiments, pseudobulbar palsy is characterized by degeneration of upper motor neurons that transmit signals to the lower motor neurons in the brain stem. In some cases, signs and symptoms include progressive loss of the ability to speak, chew, and swallow. In some instances, progressive weakness in facial muscles leads to an expressionless face. In some cases, individuals develop gravelly voice and an increased gag reflex, tongue becoming immobile and unable to protrude from the mouth. In some cases, individuals have outburst of laughing or crying.

In some embodiments, disclosed herein is a method of treating pseudobulbar palsy in a subject in need thereof, comprising administering to the subject a therapeutic effective amount of isoflupredone, an isoflupredone derivative, an isoflupredone metabolite, an isoflupredone prodrug, or a pharmaceutically acceptable salt thereof of isoflupredone. In some instances, an isoflupredone derivative comprises isoflupredone acetate, isoflupredone 21-phosphate ester disodium salt, or 21-(1-Ethoxyethoxy)-isoflupredone. In some embodiments, disclosed herein, is a method of treating pseudobulbar palsy in a subject in need thereof, comprising administering to the subject a therapeutic effective amount of isoflupredone acetate. In some embodiments, disclosed herein, is a method of treating pseudobulbar palsy in a subject in need thereof, comprising administering to the subject a therapeutic effective amount of isoflupredone 21-phosphate ester disodium salt. In some embodiments, disclosed herein, is a method of treating pseudobulbar palsy in a subject in need thereof, comprising administering to the subject a therapeutic effective amount of 21-(1-Ethoxyethoxy)-isoflupredone.

In some embodiments, disclosed herein is a method of treating pseudobulbar palsy in a subject in need thereof, comprising administering to the subject a therapeutic effective amount of 15d-PGJ₂. In some cases, also disclosed herein includes a 15d-PGJ₂ derivative, a 15d-PGJ₂ metabolite, a 15d-PGJ₂ analog, or a pharmaceutically acceptable salt thereof of 15d-PGJ₂. In some cases, disclosed herein is a method of treating pseudobulbar palsy in a subject in need thereof, comprising administering to the subject a therapeutic effective amount of a 15d-PGJ₂ derivative, a 15d-PGJ₂ metabolite, a 15d-PGJ₂ analog, or a pharmaceutically acceptable salt thereof of 15d-PGJ₂.

### Pharmaceutical Compositions/Formulations

Disclosed herein, in certain embodiments, are isoflupredone or 15d-PGJ₂ pharmaceutical compositions or formulations for treating a motor-associated neurodegenerative disease in a subject in need thereof. In some instances, also described herein include isoflupredone or 15d-PGJ₂ pharmaceutical compositions or formulations for treating a TDP-43 associated neurodegenerative disease, a C9orf72 associated neurodegenerative disease, and/or a motor neuron disease.

In some instances, pharmaceutical compositions of isoflupredone or 15d-PGJ₂ are formulated in a conventional manner using one or more physiologically acceptable carriers including excipients and auxiliaries which facilitate processing of the active compounds into preparations which are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of isoflupredone or 15d-PGJ₂ with other chemical components, such as carriers, stabilizers, diluents, dispersing agents, suspending agents, thickening agents, and/or excipients.

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The pharmaceutical formulations described herein are administered by any suitable administration route, including but not limited to, oral, parenteral (e.g., intravenous, subcutaneous, intramuscular), intranasal, buccal, topical, rectal, or transdermal administration routes.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the compositions are formulated into capsules. In some embodiments, the compositions are formulated into solutions (for example, for IV administration).

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Antifoaming agents” reduce foaming during processing which in some instances result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In some instances, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, isoflupredone, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. In some instances, “pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose are optionally used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator are optionally used in the present compositions.

The term “diluent” refers to chemical compounds that are used to dilute the compound of interest prior to delivery. Diluents are also used to stabilize compounds because they provide a more stable environment. Salts dissolved in buffered solutions (which also provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per mL, dL, or L of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol has a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants are included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

In some embodiments, the pharmaceutical compositions described herein are formulated for administration to a subject via any conventional means including, but not limited to, oral, parenteral (e.g., intravenous, subcutaneous, or intramuscular), buccal, intranasal, rectal or transdermal administration routes. In some embodiments, the pharmaceutical composition is further formulated for administration in a combined dosage form. In some embodiments, the pharmaceutical composition is formulated for administration in a separate dosage forms.

Moreover, the pharmaceutical compositions described herein, which include isoflupredone, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In some instances, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which optionally contain gum arabic, talc, polyvinylpyrrolidone, carbopol gel, polyethylene glycol, and/or titanium dioxide, lacquer solutions, and suitable organic solvents or solvent mixtures. In some instances, dyestuffs or pigments are added to the tablets or dragee coatings for identification or to characterize different combinations of active compound doses.

In some instances, pharmaceutical preparations which are used orally include push-fit capsules made of gelatin, as well as soft, sealed capsules made of gelatin and a plasticizer, such as glycerol or sorbitol. In some cases, the push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In addition, stabilizers are optionally added. All formulations for oral administration should be in dosages suitable for such administration.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations described herein are administered, for example, as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of isoflupredone, with one or more pharmaceutical excipients to form a bulk blend composition. When referring to these bulk blend compositions as homogeneous, it is meant that the particles of isoflupredone, are dispersed evenly throughout the composition so that the composition may be readily subdivided into equally effective unit dosage forms, such as tablets, pills, and capsules. In some cases, the individual unit dosages also include film coatings, which disintegrate upon oral ingestion or upon contact with diluent. In such cases, these formulations are manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

In some cases, the pharmaceutical solid dosage forms described herein include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of isoflupredone. In another embodiment, some or all of the particles of isoflupredone, are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release isoflupredone from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that are filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself optionally act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol has a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that are included in solid dosage forms described herein. The amounts of such additives are readily determined, for example, by one skilled in the art, according to the particular properties desired.

In other embodiments, one or more layers of the pharmaceutical formulation are plasticized. Illustratively, a plasticizer is generally a high boiling point solid or liquid. Suitable plasticizers are added, for example, from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating provides a delayed release of isoflupredone from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

In some instances, a capsule is prepared by placing the bulk blend of the formulation of isoflupredone, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule isopened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of isoflupredone and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with isoflupredone, which sufficiently isolate the compound of isoflupredone, from other non-compatible excipients. Materials compatible with compounds of isoflupredone, are those that delay the release of the compounds of isoflupredone, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

In some instances, microencapsulated compounds of isoflupredone are formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of compounds of isoflupredone are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds of isoflupredone are plasticized (coated) with one or more layers. Illustratively, a plasticizer is generally a high boiling point solid or liquid. Suitable plasticizers are added, for example, from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound of isoflupredone, described herein, is formulated to include one or more pharmaceutical excipients and flavors. Such a powder is prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine of the gastrointestinal tract. In some cases, the enteric coated dosage form is a compressed or molded or extruded tablet/mold (coated or uncoated) containing granules, powder, pellets, beads or particles of the active ingredient and/or other composition components, which are themselves coated or uncoated. In other cases, the enteric coated oral dosage form is a capsule (coated or uncoated) containing pellets, beads or granules of the solid carrier or the composition, which are themselves coated or uncoated.

The term “delayed release” as used herein refers to the delivery so that the release is accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile is used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH >7;

Acrylic polymers. The performance of acrylic polymers (primarily their solubility in biological fluids) varies, for example, based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. In some instances, the performance varies based on the degree and type of substitution. Cellulose acetate phthalate (CAP) dissolves in pH >6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). In some instances, the performance varies based on the degree and type of substitution. For example, HPMCP such as, HP-50, HP-55, HP-55S, HP-55F grades are suitable. In some cases, the performance varies based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH >5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) are optionally added to the coatings besides plasticizers to solubilize or disperse the coating material, and to improve coating performance and the coated product.

In other embodiments, the formulations described herein, which include isoflupredone, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of isoflupredone, described herein and at least one dispersing agent or suspending agent for oral administration to a subject. In some cases, the formulations are a powder and/or granules for suspension, and upon admixture with water, a substantially uniform suspension is obtained.

In some instances, liquid formulation dosage forms for oral administration include aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition instances, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystalline inhibitor.

In some embodiments, the aqueous suspensions and dispersions described herein remain in a homogenous state, as defined in the USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension is re-suspended into a homogenous suspension by physical agitation lasting less than 1 minute. In another embodiment, an aqueous suspension is re-suspended into a homogenous suspension by physical agitation lasting less than 45 seconds. In yet another embodiment, an aqueous suspension is re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, AvicerPH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion comprises a sweetening agent or flavoring agent in a concentration ranging from about 0.001% to about 1.0% the volume of the aqueous dispersion. In another embodiment, the aqueous liquid dispersion comprises a sweetening agent or flavoring agent in a concentration ranging from about 0.005% to about 0.5% the volume of the aqueous dispersion. In yet another embodiment, the aqueous liquid dispersion comprises a sweetening agent or flavoring agent in a concentration ranging from about 0.01% to about 1.0% the volume of the aqueous dispersion.

In addition to the additives listed above, the liquid formulations optionally include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is added just prior to administration, which ensures stability of an unstable or hydrophobic active ingredient. Thus, the SEDDS provides an effective delivery system for oral and parenteral delivery of hydrophobic active ingredients. In some instances, SEDDS provides improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that are included in formulations described herein. The amounts of such additives are readily determined by one skilled in the art, according to the particular properties desired.

### Intranasal Formulations

Intranasal formulations are known in the art and are described in, for example, U.S. Pat. Nos. 4,476,116, 5,116,817 and 6,391,452, each of which is specifically incorporated by reference. Formulations that include isoflupredone, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation described herein may be in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with isoflupredone, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which are obtained from B.F. Goodrich, is one such polymer). Other components optionally incorporated into the buccal dosage forms described herein include, but are not limited to, disintegrants, diluents, binders, lubricants, flavoring, colorants, preservatives, and the like. For buccal or sublingual administration, the compositions, for example, take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In one embodiments, the transdermal formulations described herein include at least three components: (1) a formulation of a compound of isoflupredone; (2) a penetration enhancer; and (3) an aqueous adjuvant. In addition, transdermal formulations can include additional components such as, but not limited to, gelling agents, creams and ointment bases, and the like. In some embodiments, the transdermal formulation further include a woven or non-woven backing material to enhance absorption and prevent the removal of the transdermal formulation from the skin. In other embodiments, the transdermal formulations described herein maintain a saturated or supersaturated state to promote diffusion into the skin.

Formulations suitable for transdermal administration of compounds described herein employ, for example, transdermal delivery devices and transdermal delivery patches and can be lipophilic emulsions or buffered, aqueous solutions, dissolved and/or dispersed in a polymer or an adhesive. Such patches are constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of isoflupredone. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include isoflupredone suitable for intramuscular, subcutaneous, or intravenous injection, for example, include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection also contain, e.g., additives such as preserving, wetting, emulsifying, and dispensing agents. Prevention of the growth of microorganisms utilize, for example, various antibacterial and antifungal agents, such as parabens, chlorobutanol, phenol, sorbic acid, and the like. In some instances, isotonic agents, such as sugars, sodium chloride, and the like, are also included. Prolonged absorption of the injectable pharmaceutical form can be brought about by the use of agents delaying absorption, such as aluminum monostearate and gelatin.

For intravenous injections, compounds described herein are optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include, e.g., aqueous or nonaqueous solutions, preferably with physiologically compatible buffers or excipients. Such excipients are generally known in the art.

In some instances, parenteral injections involve bolus injection or continuous infusion. Formulations for injection are presented, for example, in unit dosage form, e.g., in ampoules or in multi-dose containers, with an added preservative. In some cases, the pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the active compounds in water-soluble form. Additionally, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions contain substances which increase the viscosity of the suspension, such as sodium carboxymethyl cellulose, sorbitol, or dextran. Optionally, the suspension also contains suitable stabilizers or agents which increase the solubility of the compounds to allow for the preparation of highly concentrated solutions. Alternatively, the active ingredient is in powder form for constitution with a suitable vehicle, e.g., sterile pyrogen-free water, before use.

### Other Formulations

In certain embodiments, delivery systems for pharmaceutical compounds employ, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein is administered topically and is formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

In some instances, the compounds described herein are also formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Dosing and Treatment Regimens

In some embodiments, a pharmaceutical composition of isoflupredone or 15d-PGJ₂ described herein is administered for one or more times a day. In some embodiments, a pharmaceutical composition of isoflupredone or an isoflupredone derivative, an isoflupredone metabolite, an isoflupredone prodrug, or a pharmaceutically acceptable salt thereof of isoflupredone is administered once per day, twice per day, three times per day or more. In some cases, a pharmaceutical composition of isoflupredone or an isoflupredone derivative, an isoflupredone metabolite, an isoflupredone prodrug, or a pharmaceutically acceptable salt thereof of isoflupredone is administered daily, every day, every alternate day, five days a week, once a week, every other week, two weeks per month, three weeks per month, once a month, twice a month, three times per month, or more. In some cases, a pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, isoflupredone or an isoflupredone derivative, an isoflupredone metabolite, an isoflupredone prodrug, or a pharmaceutically acceptable salt thereof of isoflupredone is administered at a dose range of from about 0.005 mg/kg body weight to about 0.2 mg/kg body weight. In some instances, isoflupredone or an isoflupredone derivative, an isoflupredone metabolite, an isoflupredone prodrug, or a pharmaceutically acceptable salt thereof of isoflupredone is administered at a dose range of from about 0.005 mg/kg body weight to about 0.15 mg/kg body weight, from about 0.005 mg/kg body weight to about 0.1 mg/kg body weight, from about 0.005 mg/kg body weight to about 0.08 mg/kg body weight, from about 0.005 mg/kg body weight to about 0.06 mg/kg body weight, from about 0.005 mg/kg body weight to about 0.05 mg/kg body weight, from about 0.005 mg/kg body weight to about 0.04 mg/kg body weight, from about 0.005 mg/kg body weight to about 0.03 mg/kg body weight, from about 0.005 mg/kg body weight to about 0.02 mg/kg body weight, from about 0.005 mg/kg body weight to about 0.01 mg/kg body weight, from about 0.01 mg/kg body weight to about 0.15 mg/kg body weight, from about 0.01 mg/kg body weight to about 0.1 mg/kg body weight, from about 0.01 mg/kg body weight to about 0.08 mg/kg body weight, from about 0.01 mg/kg body weight to about 0.06 mg/kg body weight, from about 0.01 mg/kg body weight to about 0.05 mg/kg body weight, from about 0.01 mg/kg body weight to about 0.04 mg/kg body weight, from about 0.01 mg/kg body weight to about 0.03 mg/kg body weight, from about 0.02 mg/kg body weight to about 0.05 mg/kg weight, from about 0.03 mg/kg body weight to about 0.05 mg/kg body weight, from about 0.04 mg/kg body weight to about 0.05 mg/kg body weight, from about 0.01 mg/kg body weight to about 0.04 mg/kg body weight, from about 0.01 mg/kg body weight to about 0.03 mg/kg body weight, from about 0.01 mg/kg body weight to about 0.02 mg/kg body weight, from about 0.02 mg/kg body weight to about 0.04 mg/kg body weight, from about 0.03 mg/kg body weight to about 0.04 mg/kg body weight, or from about 0.02 mg/kg body weight to about 0.03 mg/kg body weight.

In some embodiments, isoflupredone or an isoflupredone derivative, an isoflupredone metabolite, an isoflupredone prodrug, or a pharmaceutically acceptable salt thereof of isoflupredone is administered at a dose range of from about 0.5 mg per day to about 50 mg per day. In some instances, the dose range is from about 0.5 mg per day to about 40 mg per day, from about 0.5 mg per day to about 30 mg per day, from about 0.5 mg per day to about 20 mg per day, from about 0.5 mg per day to about 15 mg per day, from about 0.5 mg per day to about 10 mg per day, from about 0.5 mg per day to about 5 mg per day, from about 1 mg per day to about 45 mg per day, from about 1 mg per day to about 40 mg per day, from about 1 mg per day to about 35 mg per day, from about 1 mg per day to about 30 mg per day, from about 1 mg per day to about 25 mg per day, from about 1 mg per day to about 20 mg per day, from about 1 mg per day to about 15 mg per day, from about 1 mg per day to about 10 mg per day, from about 1 mg per day to about 5 mg per day, from about 5 mg per day to about 50 mg per day, from about 10 mg per day to about 50 mg per day, from about 15 mg per day to about 50 mg per day, from about 20 mg per day to about 50 mg per day, from about 25 mg per day to about 50 mg per day, from about 30 mg per day to about 50 mg per day, from about 35 mg per day to about 50 mg per day, from about 40 mg per day to about 50 mg per day, from about 45 mg per day to about 50 mg per day, from about 5 mg per day to about 45 mg per day, from about 10 mg per day to about 45 mg per day, from about 15 mg per day to about 45 mg per day, from about 20 mg per day to about 45 mg per day, from about 25 mg per day to about 45 mg per day, from about 30 mg per day to about 45 mg per day, from about 35 mg per day to about 45 mg per day, from about 40 mg per day to about 45 mg per day, from about 5 mg per day to about 40 mg per day, from about 10 mg per day to about 40 mg per day, from about 15 mg per day to about 40 mg per day, from about 20 mg per day to about 40 mg per day, from about 25 mg per day to about 40 mg per day, from about 30 mg per day to about 40 mg per day, from about 35 mg per day to about 40 mg per day, from about 5 mg per day to about 35 mg per day, from about 10 mg per day to about 35 mg per day, from about 15 mg per day to about 35 mg per day, from about 20 mg per day to about 35 mg per day, from about 25 mg per day to about 35 mg per day, from about 30 mg per day to about 35 mg per day, from about 5 mg per day to about 30 mg per day, from about 10 mg per day to about 30 mg per day, from about 15 mg per day to about 30 mg per day, from about 20 mg per day to about 30 mg per day, from about 25 mg per day to about 30 mg per day, from about 5 mg per day to about 25 mg per day, from about 10 mg per day to about 25 mg per day, from about 15 mg per day to about 25 mg per day, from about 20 mg per day to about 25 mg per day, from about 5 mg per day to about 20 mg per day, from about 10 mg per day to about 20 mg per day, from about 15 mg per day to about 20 mg per day, from about 5 mg per day to about 15 mg per day, from about 10 mg per day to about 15 mg per day, or from about 5 mg per day to about 10 mg per day.

In some cases, the dose is about 0.5 mg per day, about 0.6 mg per day, about 0.7 mg per day, 0.8 mg per day, 0.9 mg per day, about 1 mg per day, about 1.5 mg per day, about 2 mg per day, about 2.5 mg per day, about 3 mg per day, about 3.5 mg per day, about 4 mg per day, about 4.5 mg per day, about 5 mg per day, about 6 mg per day, about 7 mg per day, about 8 mg per day, about 9 mg per day, about 10 mg per day, about 15 mg per day, about 20 mg per day, about 25 mg per day, about 30 mg per day, about 35 mg per day, about 40 mg per day, about 45 mg per day, or about 50 mg per day.

In some embodiments, isoflupredone or an isoflupredone derivative, an isoflupredone metabolite, an isoflupredone prodrug, or a pharmaceutically acceptable salt thereof of isoflupredone is administered as oral, parenteral (e.g., intravenous, intra-articular, subcutaneous, intramuscular, intrasynovial), intranasal, buccal, topical, rectal, or transdermal administration. In some instances, isoflupredone or an isoflupredone derivative, an isoflupredone metabolite, an isoflupredone prodrug, or a pharmaceutically acceptable salt thereof of isoflupredone is administered as a parenteral (e.g., intravenous, intra-articular, subcutaneous, intramuscular, intrasynovial) administration. In some instances, isoflupredone or an isoflupredone derivative, an isoflupredone metabolite, an isoflupredone prodrug, or a pharmaceutically acceptable salt thereof of isoflupredone is administered as an intramuscular administration. In some instances, isoflupredone or an isoflupredone derivative, an isoflupredone metabolite, an isoflupredone prodrug, or a pharmaceutically acceptable salt thereof of isoflupredone is administered as an intravenous administration. In some cases, isoflupredone or an isoflupredone derivative, an isoflupredone metabolite, an isoflupredone prodrug, or a pharmaceutically acceptable salt thereof of isoflupredone is administered as an intra-articular administration. In other cases, isoflupredone or an isoflupredone derivative, an isoflupredone metabolite, an isoflupredone prodrug, or a pharmaceutically acceptable salt thereof of isoflupredone is administered as a subcutaneous administration. In additional cases, isoflupredone or an isoflupredone derivative, an isoflupredone metabolite, an isoflupredone prodrug, or a pharmaceutically acceptable salt thereof of isoflupredone is administered as an intrasynovial administration.

In some instances, isoflupredone or an isoflupredone derivative, an isoflupredone metabolite, an isoflupredone prodrug, or a pharmaceutically acceptable salt thereof of isoflupredone is administered as a parenteral (e.g., intravenous, intra-articular, subcutaneous, intramuscular, intrasynovial) administration at a dose range of from about 0.005 mg/kg body weight to about 0.2 mg/kg body weight. In some instances, isoflupredone or an isoflupredone derivative, an isoflupredone metabolite, an isoflupredone prodrug, or a pharmaceutically acceptable salt thereof of isoflupredone is administered at a dose range of from about 0.005 mg/kg body weight to about 0.15 mg/kg body weight, from about 0.005 mg/kg body weight to about 0.1 mg/kg body weight, from about 0.005 mg/kg body weight to about 0.08 mg/kg body weight, from about 0.005 mg/kg body weight to about 0.06 mg/kg body weight, from about 0.005 mg/kg body weight to about 0.05 mg/kg body weight, from about 0.005 mg/kg body weight to about 0.04 mg/kg body weight, from about 0.005 mg/kg body weight to about 0.03 mg/kg body weight, from about 0.005 mg/kg body weight to about 0.02 mg/kg body weight, from about 0.005 mg/kg body weight to about 0.01 mg/kg body weight, from about 0.01 mg/kg body weight to about 0.15 mg/kg body weight, from about 0.01 mg/kg body weight to about 0.1 mg/kg body weight, from about 0.01 mg/kg body weight to about 0.08 mg/kg body weight, from about 0.01 mg/kg body weight to about 0.06 mg/kg body weight, from about 0.01 mg/kg body weight to about 0.05 mg/kg body weight, from about 0.01 mg/kg body weight to about 0.04 mg/kg body weight, from about 0.01 mg/kg body weight to about 0.03 mg/kg body weight, from about 0.02 mg/kg body weight to about 0.05 mg/kg weight, from about 0.03 mg/kg body weight to about 0.05 mg/kg body weight, from about 0.04 mg/kg body weight to about 0.05 mg/kg body weight, from about 0.01 mg/kg body weight to about 0.04 mg/kg body weight, from about 0.01 mg/kg body weight to about 0.03 mg/kg body weight, from about 0.01 mg/kg body weight to about 0.02 mg/kg body weight, from about 0.02 mg/kg body weight to about 0.04 mg/kg body weight, from about 0.03 mg/kg body weight to about 0.04 mg/kg body weight, or from about 0.02 mg/kg body weight to about 0.03 mg/kg body weight.

In some instances, isoflupredone or an isoflupredone derivative, an isoflupredone metabolite, an isoflupredone prodrug, or a pharmaceutically acceptable salt thereof of isoflupredone is administered as a parenteral (e.g., intravenous, intra-articular, subcutaneous, intramuscular, intrasynovial) administration at a dose range of from about 0.5 mg per day to about 50 mg per day. In some cases, the dose is about 0.5 mg per day, about 0.6 mg per day, about 0.7 mg per day, 0.8 mg per day, 0.9 mg per day, about 1 mg per day, about 1.5 mg per day, about 2 mg per day, about 2.5 mg per day, about 3 mg per day, about 3.5 mg per day, about 4 mg per day, about 4.5 mg per day, about 5 mg per day, about 6 mg per day, about 7 mg per day, about 8 mg per day, about 9 mg per day, about 10 mg per day, about 15 mg per day, about 20 mg per day, about 25 mg per day, about 30 mg per day, about 35 mg per day, about 40 mg per day, about 45 mg per day, or about 50 mg per day.

In some embodiments, a pharmaceutical composition of 15d-PGJ₂ or a 15d-PGJ₂ derivative, a 15d-PGJ₂ metabolite, a 15d-PGJ₂ analog, or a pharmaceutically acceptable salt thereof of 15d-PGJ₂ is administered once per day, twice per day, three times per day or more. In some cases, a pharmaceutical composition of isoflupredone is administered daily, every day, every alternate day, five days a week, once a week, every other week, two weeks per month, three weeks per month, once a month, twice a month, three times per month, or more. In some cases, a pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, 15d-PGJ₂ or a 15d-PGJ₂ derivative, a 15d-PGJ₂ metabolite, a 15d-PGJ₂ analog, or a pharmaceutically acceptable salt thereof of 15d-PGJ₂ is administered as oral, parenteral (e.g., intravenous, intra-articular, subcutaneous, intramuscular, intrasynovial), intranasal, buccal, topical, rectal, or transdermal administration. In some instances, 15d-PGJ₂ or a 15d-PGJ₂ derivative, a 15d-PGJ₂ metabolite, a 15d-PGJ₂ analog, or a pharmaceutically acceptable salt thereof of 15d-PGJ₂ is administered as a parenteral (e.g., intravenous, intra-articular, subcutaneous, intramuscular, intrasynovial) administration. In some instances, 15d-PGJ₂ or a 15d-PGJ₂ derivative, a 15d-PGJ₂ metabolite, a 15d-PGJ₂ analog, or a pharmaceutically acceptable salt thereof of 15d-PGJ₂ is administered as an intramuscular administration. In some instances, 15d-PGJ₂ or a 15d-PGJ₂ derivative, a 15d-PGJ₂ metabolite, a 15d-PGJ₂ analog, or a pharmaceutically acceptable salt thereof of 15d-PGJ₂ is administered as an intravenous administration. In some cases, 15d-PGJ₂ or a 15d-PGJ₂ derivative, a 15d-PGJ₂ metabolite, a 15d-PGJ₂ analog, or a pharmaceutically acceptable salt thereof of 15d-PGJ₂ is administered as an intra-articular administration. In other cases, 15d-PGJ₂ or a 15d-PGJ₂ derivative, a 15d-PGJ₂ metabolite, a 15d-PGJ₂ analog, or a pharmaceutically acceptable salt thereof of 15d-PGJ₂ is administered as a subcutaneous administration. In additional cases, 15d-PGJ₂ or a 15d-PGJ₂ derivative, a 15d-PGJ₂ metabolite, a 15d-PGJ₂ analog, or a pharmaceutically acceptable salt thereof of 15d-PGJ₂ is administered as an intrasynovial administration.

In the case wherein the patient's status does improve, upon the doctor's discretion the administration of a composition (e.g., isoflupredone or 15d-PGJ₂) is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include isoflupredone or an isoflupredone derivative, an isoflupredone metabolite, an isoflupredone prodrug, or a pharmaceutically acceptable salt thereof of isoflupredone and/or 15d-PGJ₂, or a 15d-PGJ₂ derivative, a 15d-PGJ₂ metabolite, a 15d-PGJ₂ analog, or a pharmaceutically acceptable salt thereof of 15d-PGJ₂ and optionally an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the term “treating” or “treatment” refers to the management of a subject with the intent to cure, ameliorate, stabilize, or prevent a neurodegenerative disease or condition described herein. In some instances, “treating” or “treatment” includes active treatment, or treatment directed toward the improvement of a neurodegenerative disease or condition; and also includes causal treatment, or treatment directed toward removal of the cause of the associated disease or condition. In some instances, “treating” or “treatment” include palliative treatment, or treatment designed for the relief of symptoms rather than the curing of the neurodegenerative disease or condition; preventative treatment or treatment directed to minimizing or partially or completely inhibiting the development of the associated disease or condition; and supportive treatment or treatment employed to supplement another specific therapy directed toward the improvement of the associated disease or condition. In some embodiments, “treating” and “treatment” covers any treatment of a subject, including inhibiting or ameliorating the neurodegenerative disease or condition; relieving or causing regression of the neurodegenerative disease or condition; or preventing the neurodegenerative disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### EXAMPLES

These examples are provided for illustrative purposes only and not to limit the scope of the claims provided herein.

### Example 1—Implication of a Gene Network and Isoflupredone in ALS

Unfolded protein response (UPR) is a signal transduction pathway that reestablishes homeostasis by increasing the protein folding capacity and quality control mechanism of the endoplasmic reticulum (ER). For example, stress conditions interfere with the function of ER and cause abnormal oxidative folding at the ER lumen, resulting in a condition called “ER stress”. Stress sensors within UPR, e.g., EIF2AK3, ATF6, and inositol-requiring transmembrane kinase/endonuclease (IRE1), detects accumulation of unfolded proteins and subsequently activates UPR. Eukaryotic Translation Initiation Factor 2-Alpha Kinase 3 (EIF2AK3) (also known as PRKR-Like Endoplasmic Reticulum Kinase, Pancreatic EIF2-Alpha Kinase, PERK, PEK, HsPek, and WRS) encodes a protein that phosphorylates the alpha subunit of eukaryotic translation-initiation factor 2, leading to its inactivation, and thus to a rapid reduction of translation initiation and repression of global protein synthesis. It is a type I membrane protein located in the endoplasmic reticulum (ER), where it is induced by ER stress caused by misfolded proteins. Activating Transcription Factor 6 (ATF6) (also known as CAMP-dependent transcription factor ATF-6 Alpha, Activating transcription factor 6 alpha, ATF6-Alpha and ATF6A) encodes a transcription factor that activates target genes for the unfolded protein response (UPR) during endoplasmic reticulum (ER) stress. In some instances, patients with ALS have been observed to exhibit elevated levels of EIF2AK3 and/or ATF6.

Mitochondrial biogenesis is the process by which new mitochondria are formed in the cell. In some instances, mitochondrial biogenesis is activated by numerous different signals during times of cellular stress or in response to environmental stimuli. In some cases, misregulation of genes involved in mitochondrial biogenesis leads to altered expression of proteins involved in mitochondrial homeostasis, resulting in mitochondrial damage and oxidative stress. In some instances, neuronal cells are not able to cope with mitochondrial damage and oxidative stress, leading to the pathogenesis of neurodegenerative diseases.

COP9 Signalosome Subunit 4 (COPS4) (also known as JAB1-Containing signalosome subunit 4, signalosome subunit 4, CSN4, SGN4, and COP9 constitutive photomorphogenic homolog subunit 4) encodes one of eight subunits composing COP9 signalosome, a conserved protein complex that functions as a regulator in multiple signaling pathways. In some instances, COPS4 also forms the COP9 signalosome complex (CSN) which regulates the ubiquitin (Ubl) conjugation pathway by mediating the deneddylation of the cullin subunits of SCF-type E3 ligase complexes, leading to a decrease in the Ubl ligase activity of SCF-type complexes such as SCF, CSA, or DDB2. In some instances, CSN is further involved in phosphorylation of p53/TP53, c-jun/JUN, IKBα/NFKBIA, ITPK1, IRF8/ICSBP and SNAPIN. In addition, phosphorylation of TP53 and JUN, which promotes or protects degradation by the Ubl system, respectively, has further been linked to a motor-associated neurodegenerative disease.

Tax1 (Human T-Cell Leukemia Virus Type I) Binding Protein I (TAX1BP1) (also known as TRAF6-Binding protein, T6BP, CALCOCO3 and TXBP151) encodes a HTLV-1 tax1 binding protein which interacts with TNFAIP3 to inhibit TNF-induced apoptosis. In some cases, TAX1BP1 interacts with optineurin, in which mutations within optineurin have been linked to ALS.

A gene network (termed M3 network) was identified in silico from weighted gene co-expression network analysis of public ALS gene expression datasets (GEO Accessions GSE46298—a study of a transgenic SOD1 mouse model of ALS and GSE56504—a study of laser-capture microdissected motor neurons from postmortem tissue of ALS patients). A module (termed M3 module) as related to ALS was identified by checking enrichment in genes of interest and their relation to disease. Two pathways were identified as part of the M3 module: unfolded protein response (UPR) and mitochondrial biogenesis. In addition, a set of genes as related to ALS in the presence of isoflupredone was further identified as part of the M3 module. The set of genes include EIF2AK3, ATF6, C9orf72, COPS4, and TAX1BP1. In some instances, the M3 module is decreased in ALS compared to a control (e.g., without ALS).

### Example 2—In Vitro Study of Isoflupredone in a TDP43 Stress Granule Assay

Protective effect of several compounds against cellular oxidative stress induced by arsenite was screened. The compounds were tested at 4 concentrations, along with appropriate controls (vehicle, positive and negative controls).

Reagents and Equipment

DMEM-F12 (Sigma-Aldrich D6421)

Opti-MEM (31985070)

FBS (Sigma-Aldrich F2442)

IPTG (Sigma-Aldrich I5502)

Flat bottom black 96-well plates (Becton Dickinson 353219)

Arimoclomol (Sequoia research products SRP07320a)

Sodium arsenite solution (Sigma Aldrich 35000 Fluka)

Pathway 855″ High-Content Bioimager from BD Biosciences.

Compound Dissolution

The compounds were solubilized to a final concentration of 10 mM.

Methods

Day 1: Recombinant TDP43-tGFP-U2OS cell line was thawed.

Day 2: Cells were maintained in DMEM-F12 supplemented with 10% FBS overnight at 37° C. in a humidified 5% CO2 atmosphere.

Day 3: Cells were plated in 96-well plates (approximately 5.000 cells per well). Cells were maintained in DMEM-F12 medium supplemented with 10% FBS and 5 mM IPTG for 48 h at 37° C. in a humidified 5% CO2 atmosphere.

Day 5: Cells were incubated with the test compounds at concentrations stated above for 24 hours in Opti-MEM and 5 mM IPTG.

Day 6: Cells were treated with sodium arsenite 250 μM during 120 min and TDP-43 globs formation was quantified prior to formaldehyde fixation (3.7 wt. %, 20 minutes). Nuclei were stained using DAPI (2 μg/ml) and the fluorescence was measured using a BD Pathway 855 High-Content Bioimager from Becton Dickinson. To detect DAPI, the filters used were 380/10 and 460/10 nm for excitation and emission respectively and to detect TDP43-tGFP, the filters were 488/10 nm and 515LP. The images where obtained with an objective of 20×, taking 9 pictures of each well. Cell quantification was performed delimitating the region of interest of the nuclei (stained with DAPI) using Attovision software (Becton Dickinson) and after quantification, the average of each triplicate was performed. Glob quantification was also performed using AttoVision Software. The “sub-object count” application from AttoVision Software delimitated 2 regions of interest, the nuclei and globs. The software application quantified the number of globs per cell and the average of glob number per cell of each well was calculated. After that, the average of the triplicates was performed. Both Excel 2003 and Sigmaplot 9.0. were used for data management.

An in vitro screening system was used to measure the TDP43 stress granule formation of U2OS cells expressing GFP-labeled human TDP43 in response to sodium arsenite challenge followed by drug treatment. TDP43 aggregates were detected by fluorescence imaging. The negative control was sodium arsenite (without treatment), and the positive control was arimoclomol treatment.

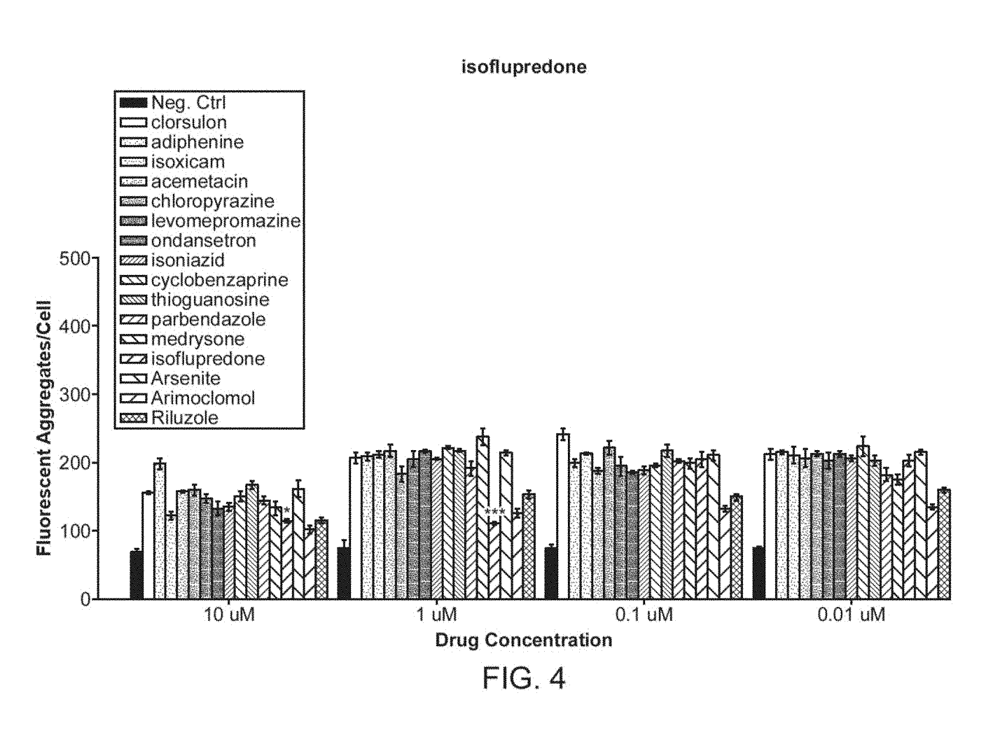

A number of drugs were tested in the TDP43 stress granules assay, including isoflupredone, medrysone, clorsulon, aiphenine, isoxicam, acemetacin, chloropyrazine, levomepromazine, ondansetron, isoniazid, cyclobenzaprine, thioguanosine, parbendazole, isoflupredone, riluzole, and arimoclomol. Isoflupredone demonstrated a statistically significant decrease in the aggregation of TDP43 in the in vitro model compared to the negative control (FIG. 4). Medrysone and other tested drugs did not show a statistically significant effect.

### Example 3—Validation of the M3 Network Activation of Isoflupredone

Bioinformatics predictions of M3 network activation from isoflupredone was validated in naïve neuronal cells (primary rat cortical neurons).

### Test Items

In parallel, 5 different test items and 2 reference items at 1 concentration each were applied to primary rat cortical neurons from P18 pups of Sprague Dawley rats. The compounds were incubated for 8 days. These cells did not receive any glutamate lesion. Cell viability was determined by MTT and RNA was isolated. A number of ≧3 technical replicates were used.

Test item 1: isoflupredone (1 micromolar)

Test item 2: 15-deoxy-delta prostaglandin J2 (10 micromolar)

Test item 3: trichostatin A (0.01 micromolar)

Test item 4: anisomycin (0.01 micromolar)

Test item 5: medrysone (0.01 micromolar)

Reference item 1: riluzole (10 micromolar)

Reference item 1: MK-801 (1 micromolar)

In all samples DMSO was included.

### Cell Culture

Primary cortical neurons were prepared from time mated Sprague Dawley rats. Animals were sacrificed and embryos were dissected in Calcium and Magnesium free Hanks Balanced Salt Solution (CMF-HBSS) containing 15 mM HEPES and 10 mM NaHCO3, pH 7.2. Embryos were decapitated, skin and skull gently removed and cortical hemispheres were separated. After removing meninges and brain stem, cortex was isolated, chopped with a sterile razor blade in Chop solution (Hibernate-E without Calcium containing 2% B-27) and digested in 2 mg/ml papain (Worthington) dissolved in Hibernate-E without Calcium for 30 minutes at 30° C. Cortices were triturated for 10-15 times with a fire-polished silanized Pasteur pipette in Hibernate-E without Calcium containing 2% B-27, 0.01% DNaseI, 1 mg/ml BSA, and 1 mg/ml Ovomucoid Inhibitor. Undispersed pieces were allowed to settle by gravity for 1 min and the supernatant was centrifuged for 2 min at 228 g. The pellet was triturated in Hibernate-E containing 2% B-27, 0.01% DNaseI, 1 mg/ml BSA, 1 mg/ml Ovomucoid Inhibitor and diluted with Hibernate-E containing 2% B-27. After the second centrifugation step, the pellet was resuspended in culture medium (Neurobasal, 2% B-27, 0.5 mM glutamine, 1% Penicillin-Streptomycin). Cells were counted in a hemacytometer and 4×10⁴ cells per well were seeded on poly-D-lysine pre-coated 96-well plates. Cells were cultured at 37° C. and 5% CO₂. Every second to third day half of the culture medium was exchanged (Neurobasal, 2% B-27 (50× stock), 0.5 mM glutamine, 1% Penicillin-Streptomycin). On Day 8, half of the culture medium was exchanged and primary cortex neurons were pre-treated with the test items at four different concentrations, the reference items at four different concentrations or with vehicle control (VC) for 24 h. The VC for all treatments was 0.1% DMSO. The test item and reference items as well as the VC were present throughout the assay, which was continued until Day 6.

### MTT Assay

Viability of cultures was determined by the MTT assay using a plate-reader (570 nm). This assay allows the measurement of the mitochondrial dehydrogenase activity which reduces yellow MTT to dark blue formazan crystals. Since this reaction was catalyzed in living cells, this assay was used for the determination of cell viability. MTT solution was added to each well in a final concentration of 0.5 mg/ml. After 2 hours the MTT containing medium was aspired. Cells were lysed in 3% SDS and the formazan crystals were dissolved in isopropanol/HCl. Optical density was measured with a plate-reader at wavelength 570 nm. Cell survival rate was expressed as optical density (OD). Values were calculated as percent of control values (vehicle control).

### RNA Sequencing

Messenger RNA was extracted from the cells and sequencing libraries were prepared using the TruSeq kit and poly-A selection. The resulting libraries were sequenced at a depth of 30 million reads per sample using single-ended 75 bp reads.

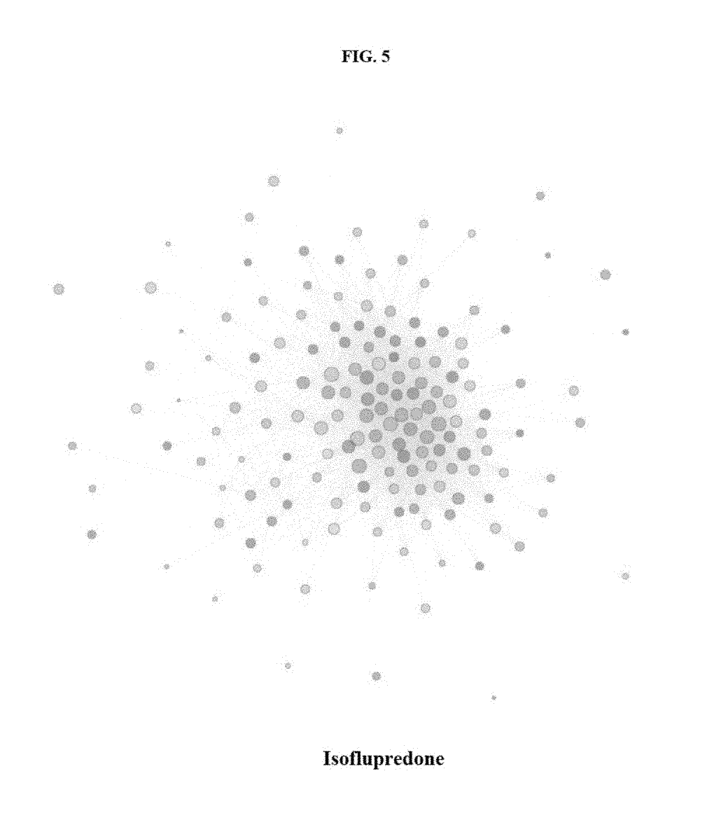

FIG. 5 shows the activation of M3 network genes in response to isoflupredone (1 μM) in primary rat cortical neurons. The pattern of activation is largely up-regulation (red) with only sparse down-regulation (green), consistent with the predicted effect.

### Example 4—Pharmacokinetic Studies of Isoflupredone Acetate

The goal of the studies are to evaluate the PK of isoflupredone acetate in Sprague-Dawley rats and C57Bl/6 mice following a single intravenous bolus (iv; 1 mg eq./kg), oral gavage (po; 1 and 10 mg eq./kg), or subcutaneous (sc; 1 and 10 mg eq./kg) administration.

Animals and Housing

Sprague Dawley (SD) rats (138 adult males), and C57Bl/6 mice (140 adult males), are obtained (Hilltop Laboratories, Scottsdale, Pa.); 123 per species are used in the study and the remainder provided blank matrix. Animals are individually identified by tail markings and are acclimated to the study environment for 16-21 days prior to dose administration. Animals are individually housed in suspended wire caging, and are kept on a 12h/12h light/dark cycle except when interrupted for study procedures. Average room temperature is regulated in the range 18 to 29° C., average relative humidity of 30-70%, and an average daily airflow >10 fresh air changes/h. Animals are fed LabDiet Certified Rodent Diet 5002 Meal food ad libitum, except during fasting prior to dose administration, and had access to water ad libitum.

Test Article and Formulation Preparation

Isoflupredone acetate is dosed as a solution in physiological saline as a single dose via intravenous bolus (1 mg eq./kg), oral gavage (po; 1 and 10 mg eq./kg), or subcutaneous (sc; 1 and 10 mg eq./kg) administration. The concentration of isoflupredone acetate in the 1 mg eq./kg iv, po, and sc solutions dosed to rats is 1 mg eq./mL, and the dose volume is 1 mL/kg. The concentration of isoflupredone acetate for the same dose level administered to mice is 0.2 mg eq./mL, and the dose volume is 5 mL/kg. The concentration of isoflupredone acetate in the 10 mg eq./kg po and sc solutions is 5 mg eq./mL and the dose volume is 2 mL/kg for both rats and mice. Blood (for preparation of plasma) and brain samples are collected at 0.08, 0.25, 0.5, 1, 2, 4, 8, and 24h post-dose.

Pharmacokinetic Analysis

Since all time points are terminal, mean isoflupredone concentrations per time point are used to calculate the composite PK parameters by non-compartmental analysis using WinNonlin program, version 5.2 (Pharsight Corp., Mountain View, Calif.). A model is selected based on the vascular (iv bolus) or extravascular (po or sc) routes of administration. For the iv route, plasma concentration at time zero is back extrapolated from the first two observed post dose plasma concentrations. For the po and sc routes, concentration at time zero is assumed to be zero. Plasma and tissue concentrations below the limit of quantitation are treated as absent samples for the purpose of calculating the mean plasma concentration values or for calculating PK parameters.

The area under the plasma concentration versus time curve (AUC) is calculated using the linear trapezoidal method (linear interpolation). When appropriate, the terminal elimination phase of the PK profile is estimated based on the best fit (r²) using at least the last three observed concentrations. PK parameters describing the systemic exposure of isoflupredone acetate in plasma and brain are estimated from observed (rather than predicted) plasma/brain concentrations, the dosing regimen, the AUC, and the terminal elimination phase rate constant (k ei) for each group. The portion of the AUC from the last measurable concentration to infinity is estimated from the equation Ct/kₑₗ, where Ct represents the last measurable concentration. The extrapolated portion of the AUC is used for the determination of AUC_{0-∞}. The percent bioavailability (% F) is calculated by dividing the dose normalized extravascular plasma AUC_{0-∞}by the dose normalized iv plasma AUC₀₋₂₈ times 100. The bioavailability calculations assumed concentrations are in the linear range.

Bioanalysis

Concentrations of isoflupredone acetate in rat and mouse plasma and brain are determined using an LC-MS/MS assay developed at ABC Laboratories. Calibration standards are prepared by adding isoflupredone acetate to control plasma or control brain homogenate obtained from Sprague-Dawley rats or C57Bl/6 mice. Three calibration curves are prepared using concentrations from 1.00-5000 ng eq./mL for plasma, 0.500-2500 ng eq./mL for brain (1 and 1.22 mg eq/kg doses) and 1.00-5000 ng eq./mL for brain (10 mg eq/kg doses).

### Example 5—Clinical Trial

The objective of this study will be to evaluate the safety, tolerability, and effect of Isoflupredone Acetate in patients with amyotrophic lateral sclerosis.

Study Type: Interventional

Study Design: Endpoint Classification: Safety/Efficacy Study

Intervention Model: Single Group Assignment

Masking: Open Label

Primary Purpose: Treatment

Primary Outcome Measures: Protein in the cerebrospinal fluid [Time Frame: baseline, week 12, end of study] [Designated as safety issue: No]

Secondary Outcome Measures: Adverse Events [Time Frame: Week 3, 6, 9, 12, 15, 18, 24, 30, & 36 visits] [Designated as safety issue: No]

Eligibility:

Ages Eligible for Study: 18 Years and older

Genders Eligible for Study: Both

Accepts Healthy Volunteers: No

Criteria:

Inclusion Criteria:

Subjects with definite, probable, or laboratory supported probable ALS will be eligible.

ALS diagnosed as probable, laboratory supported probable or definite according to the World Federation of Neurology El Escorial criteria [Brooks et al. 2000]

Age 18 or older

Capable of providing informed consent and complying with trial procedures

Not taking Riluzole (Rilutek) or on a stable dose for 30 days

Not taking Coenzyme QR10R or on a stable dose and brand for 30 days

Absence of exclusion criteria

Exclusion Criteria:

History or evidence of malabsorption syndromes

Exposure to any experimental agent within 30 days of onset of this protocol

Women who are pregnant or planning to become pregnant

Women of childbearing potential not practicing contraception

Women who are breastfeeding

Enrollment in another research study within 30 days of or during this trial

Alcoholism

Patients taking phenytoin (Dilantin) or other therapy affecting folate levels

Dementia (MMSE <22)

Seizure disorder

Folate deficiency

Megaloblastic anemia

Cardiovascular disorder/arrhythmia

Impaired kidney function, defined as creatinine levels of 2.5×ULN

Impaired liver function, defined as AST or ALT of 3×ULN

Advanced ALS patients, defined as those with any of the following: forced vital capacity <60% (use of BIPAP is allowed); tracheostomy; or mechanical ventilation

Use of any of the following medications: cytosine, arabinoside, methotrexate, daunorubicin, sulfonamides, zidovudine, lorazepam, coumadin, sulfamethoxazole, and trimethoprim

Patients taking Lithium within 30 days of or during this trial

Incapable of providing informed consent and complying with trial procedures

### Example 6—In Vitro Study of 15d-PGJ₂ in a TDP43 Stress Granule Assay

Protective effect of several compounds including 15d-PGJ₂ against cellular oxidative stress induced by arsenite was screened. The compounds were tested following the protocol as illustrated in Example 2.

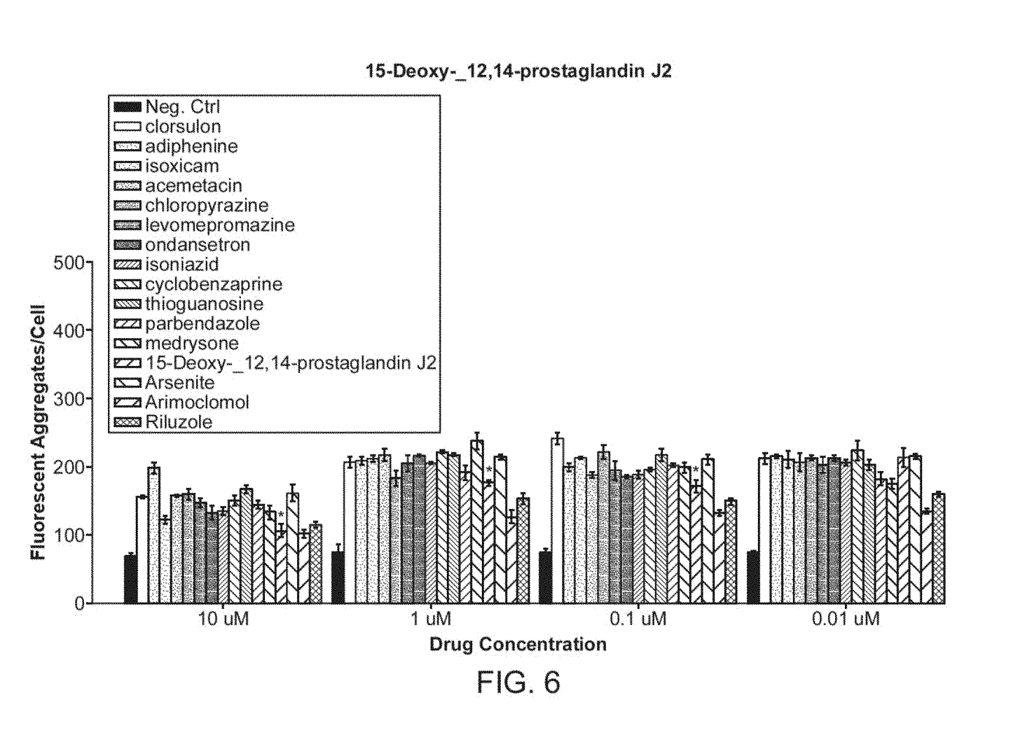

Administration of 15d-PGJ₂ in primary cortex neurons showed a decrease in the aggregation of TDP-43 (FIG. 6). Additional drugs tested did not show a statistically significant decrease.

### Example 7—In Vitro Study of 15d-PGJ₂ in a Glutamate Excitotoxicity Model

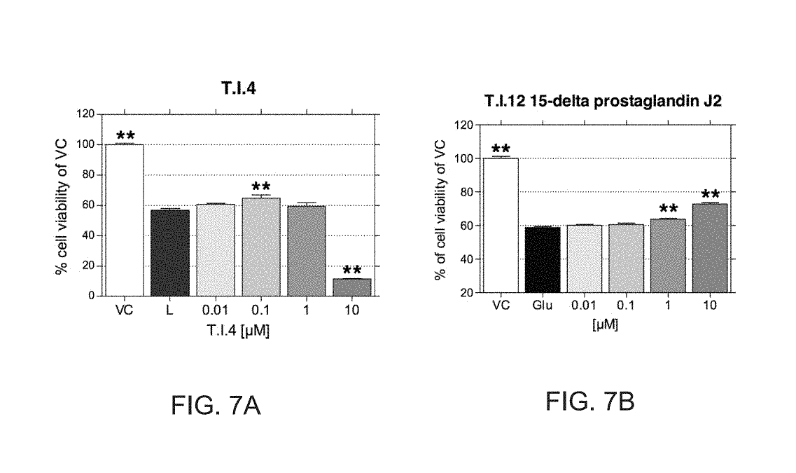

15d-PGJ₂ was tested in a glutamate excitotoxicity model, a model of ALS. 15d-PGJ₂ was incubated with glutamate excitotoxicity for 24 hours in two cell models, NSC-34 cells (which are derived from mouse motor neurons), and primary rat cortical neurons. As illustrated in FIG. 7, 15d-PGJ₂ exerted a neuroprotective effect in both cell lines.

### Example 8

The following table illustrates the sequence of TDP-43 protein (Accession No. NP_031401.1)

| | | SEQ |
|---|---|---|
| | | ID |
| Name | Sequence | NO: |
| TDP-43 | MSEYIRVTEDENDEPIEIPSEDDGTVLLSTVTAQFPGAC | 1 |
| | GLRYRNPVSQCMRGVRLVEGILHAPDAGWGNLVYV | |
| | VNYPKDNKRKMDETDASSAVKVKRAVQKTSDLIVLG | |
| | LPWKTTEQDLKEYFSTFGEVLMVQVKKDLKTGHSKG | |
| | FGFVRFTEYETQVKVMSQRHMIDGRWCDCKLPNSKQ | |
| | SQDEPLRSRKVFVGRCTEDMTEDELREFFSQYGDVM | |
| | DVFIPKPFRAFAFVTFADDQIAQSLCGEDLIIKGISVHIS | |
| | NAEPKHNSNRQLERSGRFGGNPGGFGNQGGFGNSRG | |
| | GGAGLGNNQGSNMGGGMNFGAFSINPAMMAAAQA | |
| | ALQSSWGMMGMLASQQNQSGPSGNNQNQGNMQRE | |
| | PNQAFGSGNNSYSGSNSGAAIGWGSASNAGSGSGFNG | |
| | GFGSSMDSKSSGWGM | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of treating a motor-associated neurodegenerative disease in a subject in need thereof, comprising administering to the subject a therapeutic effective amount of isoflupredone, an isoflupredone derivative, an isoflupredone metabolite, an isoflupredone prodrug, or a pharmaceutically acceptable salt of isoflupredone.

2. The method of claim 1, wherein the method comprises administering to the subject a therapeutic effective amount of isoflupredone.

3. The method of claim 1, wherein the isoflupredone derivative comprises isoflupredone acetate, 21-(1-Ethoxyethoxy)-isoflupredone or isoflupredone 21-phosphate ester disodium salt.

4. The method of claim 1, wherein the isoflupredone derivative comprises isoflupredone acetate.

5. The method of claim 1, wherein the motor-associated neurodegenerative disease is a TDP-43 associated neurodegenerative disease.

6. The method of claim 1, wherein the motor-associated neurodegenerative disease is a C9orf72 associated neurodegenerative disease.

7. The method of claim 1, wherein the motor-associated neurodegenerative disease comprises a motor neuron disease.

8. The method of claim 7, wherein the motor neuron disease comprises amyotrophic lateral sclerosis, primary lateral sclerosis, progressive muscular atrophy, progressive bulbar palsy, or pseudobulbar palsy.

9. The method of claim 1, wherein the motor-associated neurodegenerative disease is amyotrophic lateral sclerosis.

10. The method of claim 1, wherein the motor-associated neurodegenerative disease is frontotemporal dementia.

11. The method of claim 1, wherein isoflupredone, an isoflupredone derivative, an isoflupredone metabolite, an isoflupredone prodrug, or a pharmaceutically acceptable salt of isoflupredone is formulated for parenteral administration.

12. The method of claim 11, wherein isoflupredone, an isoflupredone derivative, an isoflupredone metabolite, an isoflupredone prodrug, or a pharmaceutically acceptable salt of isoflupredone is formulated for intravenous, intramuscular, or subcutaneous administration.

13. The method of claim 1, wherein the subject is diagnosed with a motor-associated neurodegenerative disease.

14. The method of claim 1, wherein the subject has a mutation in one or more of EIF2AK3, ATF6, C9orf72, SOD1, TARDBP, FUS, COPS4, or TAX1BP1.

15. The method of claim 1, wherein the subject has an elevated level of EIF2AK3 and/or ATF6 relative to a control subject.

16. The method of claim 15, wherein the control subject is a subject with a normal level of EIF2AK3 and/or ATF6.

17. The method of claim 1, wherein the treatment with isoflupredone, an isoflupredone derivative, an isoflupredone metabolite, an isoflupredone prodrug, or a pharmaceutically acceptable salt of isoflupredone modulates unfolded protein response pathway or mitochondrial biogenesis pathway.

18. The method of claim 1, wherein the subject is human.

19. A method of treating a TDP-43 associated neurodegenerative disease in a subject in need thereof, comprising administering to the subject a therapeutic effective amount of isoflupredone, an isoflupredone derivative, an isoflupredone metabolite, an isoflupredone prodrug, or a pharmaceutically acceptable salt of isoflupredone.

20. A method of treating amyotrophic lateral sclerosis in a subject in need thereof, comprising administering to the subject a therapeutic effective amount of isoflupredone, an isoflupredone derivative, an isoflupredone metabolite, an isoflupredone prodrug, or a pharmaceutically acceptable salt of isoflupredone.
